(19) United States
(12) Patent Application Publication
(10) Pub. No.: US 2026/0284410 A1
(43) Pub. Date: Sep. 24, 2026

(54) MEDICAL DEVICE SYSTEM AND METHOD FOR DELIVERING CONDUCTION SYSTEM PACING

(71) Applicant: Medtronic, Inc., Minneapolis, MN (US)

(72) Inventors: Robert W. STADLER, Shoreview, MN (US); Hongyang LU, Shanghai (CN); Tianyi SHI, Shanghai (CN)

(21) Appl. No.: 19/490,210

(22) PCT Filed: Jun. 9, 2023
(86) PCT No.: PCT/CN2023/099308

§ 371 Date: Dec. 4, 2025

(51) Int. Cl. A61N 1/365; A61N 1/02; A61N 1/37; A61N 1/372

(52) U.S. Cl. CPC A61N 1/365; A61N 1/025; A61N 1/3706; A61N 1/371; A61N 1/37252

(57) ABSTRACT

A medical device is configured to receive a conduction system pacing therapy command which may correspond to one of a first conduction system pacing therapy command or a second conduction system pacing therapy command. The medical device may determine an atrioventricular (AV) pacing interval according to a first method in response to receiving the first conduction system pacing therapy command. The medical device may determine the AV pacing interval according to a second method different than the first method in response to receiving the second conduction system pacing therapy command. The medical device may schedule conduction system pacing pulses for delivery at the determined AV pacing interval.

### TECHNICAL FIELD

This disclosure relates to a medical device system for delivering cardiac pacing according to a conduction system pacing (CSP) therapy.

### BACKGROUND

During normal sinus rhythm (NSR), the heartbeat is regulated by electrical signals produced by the sino-atrial (SA) node located in the right atrial wall. Each depolarization signal produced by the SA node spreads across the atria, causing the depolarization and contraction of the atria, and arrives at the atrioventricular (AV) node. The AV node responds by propagating a depolarization signal through the bundle of His of the atrioventricular septum and thereafter to the left and right bundle branches and the Purkinje muscle fibers of the right and left ventricles, sometimes referred to as the “His-Purkinje system” and referred to herein as the “conduction system.”

Patients with poor SA node function, poor AV node conduction (sometimes referred to as AV block), or conduction system abnormalities of the His bundle or left and/or right bundle branches (sometimes referred to as bundle branch block) or other conduction system abnormalities may receive a pacemaker to restore a more normal heart rhythm and heart chamber synchrony. Atrial pacing may be performed to provide a regular atrial rate in a patient having SA node dysfunction. Ventricular pacing may be performed to provide a regular ventricular rate in a patient having AV conduction abnormalities. A single chamber ventricular pacemaker may be coupled to a transvenous ventricular lead carrying electrodes placed in the right ventricle (RV), e.g., in the right ventricular apex. The pacemaker itself is generally implanted in a subcutaneous pocket with the transvenous ventricular lead tunneled to the subcutaneous pocket. Intracardiac pacemakers have been introduced or proposed for implantation entirely within a patient's heart, eliminating the need for transvenous leads. An intracardiac pacemaker may provide sensing and pacing from within a chamber of the patient's heart, e.g., from within the right ventricle in a patient having AV conduction block.

Dual chamber pacemakers are available which include a transvenous atrial lead carrying electrodes which are placed in the right atrium and a transvenous ventricular lead carrying electrodes that are placed in the right ventricle via the right atrium. A dual chamber pacemaker senses atrial electrical signals and ventricular electrical signals and can provide both atrial pacing and ventricular pacing as needed to promote a normal atrial and ventricular rhythm and promote AV synchrony when SA and/or AV node or other conduction abnormalities are present.

### SUMMARY

The techniques of this disclosure generally relate to a medical device and methods for delivering and controlling cardiac pacing according to different types of CSP therapies. An implantable medical device (IMD), e.g., a pacemaker or implantable cardioverter defibrillation (ICD), can be configured to receive a CSP therapy command transmitted from an external device. The CSP therapy command may be selected by a user and entered via a user interface of the external device. The IMD is configured to enable or disable AV conduction monitoring and enable or disable different pacing modes based on the received CSP therapy command. Furthermore, the IMD is configured to select or determine an AV pacing interval used to control the timing of CSP pulses based on the CSP therapy programmed by the CSP therapy command. The CSP pulses may be delivered at one or more CSP sites along the His-Purkinje system. In some examples, the CSP pulses are delivered in the area of the left bundle branch (LBB), which can be referred to as LBB area pacing (LBBAP), the area of the right bundle branch (RBB), which can be referred to as RBBAP, and/or the area of the His bundle.

In one example, the disclosure provides a medical device including a sensing circuit configured to sense at least one cardiac electrical signal and a therapy delivery circuit configured to generate atrial pacing pulses and CSP pulses. The medical device may further include a communication circuit configured to receive CSP therapy command from among at least a first CSP therapy command or a second CSP therapy command. The medical device includes a control circuit configured to receive the CSP therapy command from the communication circuit, wherein the CSP therapy command may correspond to one of the first CSP therapy command or the second CSP therapy command. The control circuit may be further configured to determine an AV pacing interval according to a first method when the CSP therapy command is the first CSP therapy command and determine the AV pacing interval according to a second method different than the first method when the CSP therapy command is the second CSP. The control circuit may schedule conduction system pacing pulses for delivery by the therapy delivery circuit at the determined AV pacing interval and according to the received one of the first CSP therapy command or the second CSP therapy command.

In another example, the disclosure provides a method including sensing at least one cardiac electrical signal, receiving one of a first CSP therapy command or a second CSP therapy command and determining an AV pacing interval according to a first method in response to receiving the first CSP therapy command and determining the AV pacing interval according to a second method different than the first method in response to receiving the second CSP therapy command. The method may further include scheduling CSP pulses for delivery at the determined AV pacing interval and according to the received one of the first CSP therapy command or the second CSP therapy command.

In another example, the disclosure provides a non-transitory computer readable medium storing instructions which, when executed by a control circuit of a medical device, cause the medical device to sense at least one cardiac electrical signal and receive a CSP therapy command from among at least a first CSP therapy command or a second CSP therapy command. The instructions may further cause the medical device to determine an AV pacing interval according to a first method in response to receiving the first CSP therapy command and determine the AV pacing interval according to a second method different than the first method in response to receiving the second CSP therapy command. The instructions may further cause the medical device to schedule CSP pulses for delivery at the determined AV pacing interval and according to the received one of the CSP therapy command or the second CSP therapy command.

In another example, the disclosure provides a medical device system including a user interface configured to receive a user input CSP therapy command and display an auto-populated pacing mode field based on the received user input CSP therapy command. The medical device system may further include a communication circuit for transmitting the user input CSP therapy command to an implantable medical device configured to deliver a CSP therapy according to the CSP therapy command. In some examples, the CSP therapy command may be a minimize CSP therapy command, a maximize CP therapy command or a complete AV block therapy command.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1A is a conceptual diagram of a medical device system for sensing cardiac electrical signals and delivering CSP therapies according to some examples.

FIG. 1B is a conceptual diagram of a leadless intracardiac pacemaker positioned within the right atrium for providing CSP in the area of the His bundle according to some examples.

FIG. 2 is a conceptual diagram of circuitry that may be enclosed within an IMD configured to deliver CSP therapies and sense cardiac electrical signals according to the techniques disclosed herein.

FIG. 3 is a flow chart of a method performed by an IMD for controlling CSP therapy according to some examples.

FIG. 4 is a flow chart that is a continuation of the flow chart of FIG. 3 of a method for controlling CSP therapy delivery by an IMD according to some examples.

FIG. 5 is a flow chart of a method for delivering CSP during an atrial asynchronous CSP mode in response to detecting an atrial tachyarrhythmia.

FIG. 6 is a flow chart of a method for performing asynchronous CSP during an atrial tachyarrhythmia according to another example.

FIG. 7 is a diagram of a method for delivering asynchronous CSP during a detected atrial tachyarrhythmia to promote CSP and capture of the ventricles at a regular ventricular rate.

FIG. 8 is diagram of an example user interface CSP therapy programming window that may be displayed by an external device for enabling a user to select a CSP therapy command for transmission to an IMD.

FIG. 9 is a diagram of a user interface programming window that may be displayed by an external medical device when the CSP therapy is selected to be the minimize CSP therapy.

FIG. 10 is a diagram of a user interface programming window that may be displayed by an external device when the CSP therapy is selected to be the complete AV block therapy.

FIG. 11 is a diagram of pacing mode operating states of a control circuit of an IMD operating according to a received CSP therapy command according to some examples.

### DETAILED DESCRIPTION

A medical device system capable of delivering different types of CSP therapies to improve electrical synchrony of the heart is disclosed herein. Ventricular pacing via electrodes at or near the right ventricular apex has been found to be associated with increased risk of atrial fibrillation and heart failure. Alternative pacing sites along the His-Purkinje conduction system may provide a more physiological electrical activation of the heart along the heart's native conduction system. Pacing the ventricles via the His bundle, the RBB and/or the LBB for example, allows recruitment along the heart's natural conduction system, including the Purkinje fibers. An IMD as disclosed herein is configured to receive a CSP therapy command transmitted by a programmer or other external device and controls the delivery of cardiac pacing based on the CSP therapy command received.

As used herein, the term “CSP therapy” refers to cardiac pacing delivered according to one or more pacing modes to at least one CSP site, e.g., the area of the His bundle, LBB, RBB, Purkinje fibers, or any combination thereof for causing ventricular depolarization. The CSP therapy may be selected by a clinician or other user of an IMD programmer or other external device of an IMD system. The CSP therapy may be selected by the clinician or other user based generally on a cardiac conduction condition of the patient as further described below. In some examples, the CSP therapy selected by the user and a corresponding CSP therapy command transmitted to the IMD may be selected from a minimum CSP therapy, maximum CSP therapy and a complete AV block therapy. As further described below, the IMD may be configured to enable two or more pacing modes and automatic switching between the two or more pacing modes for delivering the selected CSP therapy in accordance with the received CSP therapy command. The IMD may be configured to enable a primary or permanent pacing mode based on the CSP therapy command, determine or store an AV pacing interval used in scheduling CSP pulses based on the received CSP therapy command, and enable or disable AV conduction checks based on the received CSP therapy command.

The pacing modes enabled by the IMD based on the CSP therapy command may be designated by a three or four letter notation where the first letter can indicate the heart chamber(s) being paced (e.g., A for atria, V for ventricles, D for dual chamber pacing of both the atria and ventricles or O for no pacing). The second letter can indicate the heart chamber being sensed (e.g., A for atrial sensing, V for ventricular sensing, D for dual chamber sensing in both the atria and the ventricles or O for no sensing). The third letter can stand for the pacing response of the IMD to a sensed cardiac event signal (e.g., a P-wave attendant to atrial depolarization or an R-wave attendant to ventricular depolarization). The response may be I for inhibit a pacing pulse in response to the sensed cardiac event signal, T for triggering a pacing pulse in response to the sensed cardiac event signal, D for both triggering and inhibiting pacing pulses in response to sensed cardiac event signals, or O for no pacing response to sensed cardiac event signals. The fourth letter, when present, may represent rate response pacing by the letter R in which case the IMD may automatically adjust the pacing rate to provide increased pacing rate support during increased patient physical activity.

For example, a DDD(R) pacing mode may indicate pacing in both the atria and ventricles (via the conduction system), sensing in both the atrial and the ventricles, and a dual pacing response. The dual pacing response can include inhibiting atrial pacing pulses in response to sensed atrial event signals (e.g., P-waves), inhibiting ventricular pacing pulses in response to sensed ventricular event signals (e.g., R-waves) and triggering ventricular pacing pulses in response to a sensed atrial event signal for delivering a ventricular pacing pulse (at a CSP site) at an AV pacing interval following a sensed atrial event signal. The “R” when present can indicate rate response pacing during increased patient physical activity, which can be sensed by an activity sensor such as an accelerometer. A single chamber pacing mode may be designated, for example, as an AAI or ADI pacing mode with pacing delivered only in the atrial chamber, with single chamber atrial sensing or dual chamber sensing, and atrial pacing pulses inhibited in response to sensed P-waves.

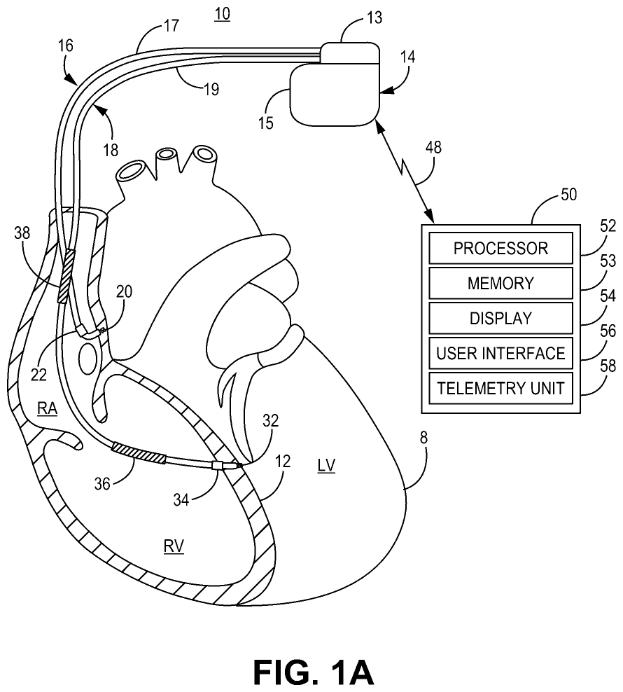

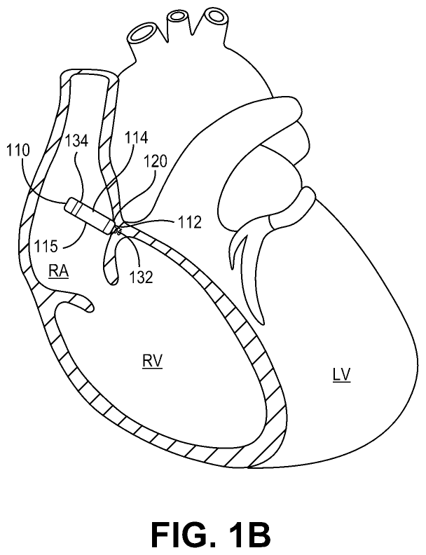

FIG. 1A is a conceptual diagram of a medical device system 10 for sensing and analyzing cardiac electrical signals and delivering CSP therapies according to some examples. Medical device system 10 includes a pacemaker 14 connected to a right atrial (RA) pacing lead 16 and a CSP lead 18 in this example. Pacemaker 14 includes a housing 15, which may be hermetically sealed, to enclose internal circuitry corresponding to the various circuits and components for sensing cardiac signals from heart 8 and controlling CSP therapy delivered to heart 8 by pacemaker 14. The housing 15 may be formed of a conductive material, such as titanium or titanium alloy. The housing 15 may function as an electrode (sometimes referred to as a “can” electrode). In some examples, housing 15 may be available as a return anode electrode delivering unipolar pacing pulses and/or for use in a sensing electrode vector for sensing cardiac electrical signals in combination with electrodes carried by lead 16 and/or lead 18.

Pacemaker 14 includes a connector block 13 coupled to housing 15 including connector bores configured to receive the proximal lead connectors (not shown) of RA lead 16 and CSP lead 18. Connector block 13 may have one or more additional connector bores for receiving one or more additional leads, e.g., for receiving a coronary sinus lead for providing pacing and sensing in the left ventricle (LV) of heart 8 in some examples.

RA lead 16 is shown advanced transvenously into the right atrium of a patient's heart 8 for sensing atrial signals, e.g., P-waves attendant to atrial depolarizations, and for delivering atrial pacing pulses. RA lead 16 includes pacing and sensing electrodes 20 and 22. Electrode 20 is shown as a screw-in, helical tip electrode at the distal end of RA lead 16. Electrode 22 is shown as a ring electrode spaced proximally from tip electrode 20. Electrodes 20 and 22 can form a bipolar pair for sensing atrial signals and delivering atrial pacing pulses via tip electrode 20 as a cathode electrode and ring electrode 22 as the return anode electrode. RA lead 16 includes an elongated lead body 17 through which insulated electrical conductors extend from the respective electrodes 20 and 22 to a proximal lead connector (not shown) connected to the pacemaker 14 via connector assembly 13. The electrodes 20 and 22 are thereby connected to internal electronics of pacemaker 14 via respective electrical feedthroughs in connector assembly 13 that cross pacemaker housing 15.

CSP lead 18 is shown advanced transvenously into the right atrium of a patient's heart 8 and further into the right ventricle for positioning tip electrode 32 within the interventricular septum in the vicinity of the heart's conduction system, e.g., at an LBBAP site or at an RBBAP site. CSP lead 18 is positioned for sensing ventricular signals, e.g., R-waves attendant to intrinsic and/or pacing evoked ventricular depolarizations, and for delivering CSP pulses. CSP lead 18 includes pacing and sensing electrodes 32 and 34. Electrode 32 is shown as a screw-in, helical tip electrode at the distal end of CSP lead 18. Electrode 34 is shown as a ring electrode spaced proximally from tip electrode 32. Electrodes 32 and 34 can form a bipolar pair for sensing ventricular signals and delivering CSP pulses via tip electrode 32 as a cathode electrode and ring electrode 34 as the return anode electrode. While the electrodes 20, 22, 32 and 34 are represented as helical screw-in electrodes or ring electrodes in FIG. 1A, it is to be understood that other electrode types may be used such as button electrodes, hook electrodes, segmented electrodes, short coil electrodes, or the like.

In some examples, pacemaker 14 may be capable of delivering cardioversion/defibrillation (CV/DF) shocks for treating ventricular tachyarrhythmias. In this case, CSP lead 18 (and/or another lead coupled to pacemaker 14) may carry one or more coil electrodes 36 and 38 for use in delivering high voltage CV/DF shocks. As such, while pacemaker 14 is referred to as a “pacemaker” herein, it could be referred to as “implantable cardioverter defibrillator” or “ICD” when capable of delivering high voltage CV/DF shocks in addition to the pacing functionality as disclosed herein.

CSP lead 18 includes an elongated lead body 19 through which insulated electrical conductors extend from the respective electrodes 32 and 34 (and coil electrodes 36 and 38 if present) to a proximal lead connector (not shown) connected to the pacemaker 14 via connector assembly 13. The electrodes 32 and 34 (and coil electrodes 36 and 38 if present) are thereby connected to internal electronics of pacemaker 14 via respective electrical feedthroughs in connector assembly 13 that cross pacemaker housing 15.

Electrodes 20, 22, 32, 34 (and 36 and 38 if present) may be formed from titanium, platinum, iridium or alloys thereof, as examples with no limitation intended, and may include a low polarizing coating, such as titanium nitride, iridium oxide, ruthenium oxide, platinum black, among others. Lead bodies 17 and 19 may each be formed from a non-conductive material, including silicone, polyurethane, fluoropolymers, mixtures thereof, and/or other appropriate materials. Each lead body may be shaped to form one or more lumens within which one or more insulated electrical conductors extend between the proximal lead connectors and the distal electrodes 20, 22, 32, 34, 36 and 38 carried by the respective lead 16 or 18.

While CSP lead 18 is shown advanced into the RV for positioning tip electrode 32 in the interventricular septum in the area of the LBB or the RBB for delivering CSP, it is to be understood that the distal end of CSP lead 18 may be positioned at other locations for delivering CSP to heart 8 for causing depolarization of the ventricles and corresponding ventricular systole. For instance, CSP lead tip electrode 32 may be positioned along or in the area of the His bundle and/or the RBB or LBB in a basal portion of the interventricular septum. In other examples, CSP lead 18 may be advanced into the RA with tip electrode 32 advanced into the interatrial septum toward the His bundle, e.g., at the inferior end of the interatrial septum. CSP lead 18 may be advanced toward the His bundle from a location beneath the AV node and near the tricuspid valve annulus, generally in the Triangle of Koch, to position CSP lead tip electrode 32 near the His bundle from a right atrial approach. The techniques disclosed herein are not limited to a particular CSP location and may be practiced in a variety of IMD systems including at least one electrode that can be positioned at a CSP site, including leadless pacemakers and/or pacemakers or ICDs coupled to one or more leads. Other examples of IMDs configured to deliver CSP at various example CSP sites that may be adapted to perform the techniques disclosed herein are generally disclosed in U.S. Patent Application Publication No. 2022/0362558 (Zhou, et al., filed May 3, 2022) and in U.S. Pat. No. 11,607,550 (Cao, et al., filed on Jun. 15, 2020), the content both of which incorporated herein by reference in its entirety.

As described below, cardiac electrical signal sensing circuitry included in pacemaker 14 may receive a cardiac electrical signal sensed from electrodes carried by RA lead 16 and a cardiac electrical signal sensed from electrodes carried by CSP lead 18 for use in controlling the timing and delivery of atrial pacing pulses and CSP pulses according to a programmed CSP therapy. Pacemaker 14 includes therapy delivery circuitry for generating pacing pulses delivered via the RA lead 16 and CSP lead 18.

Medical device system 10 is shown including an external medical device 50 for receiving data from pacemaker 14 and for transmitting programming commands to pacemaker 14 corresponding to a user-selected CSP therapy command and various sensing and pacing control parameters used by pacemaker 14. For instance, during pacemaker implantation, during clinical follow-up procedures, and/or during remote monitoring of the patient, a user interacting with external device 50 may select a CSP therapy command for transmission to pacemaker 14. Pacemaker 14 may enable or disable AV conduction checks, enable and/or disable one or more pacing modes, and determine and/or store AV pacing intervals used for controlling atrial synchronous CSP pulses based on a received CSP therapy command.

As further described below, the CSP therapy command received by pacemaker 14 from external device 50 may determine the pacing modes that may be enabled and applied by pacemaker 14 and the manner in which pacemaker 14 controls delivery of CSP pulses to heart 8. For example, depending on a cardiac conduction condition of the patient, a clinician may program the CSP therapy to be a minimum CSP therapy, a maximum CSP therapy, or a complete AV block therapy, all of which are described below in conjunction with the various flow charts and diagrams presented herein. In a patient presenting relatively normal ventricular synchrony but experiencing intermittent or occasional AV conduction block that can require ventricular pacing, the clinician may program a minimum CSP therapy. This CSP therapy may be referred to as “minimum CSP” because intrinsic AV conduction is promoted when present, and CSP can be provided when AV conduction block or a long AV conduction time is detected.

In a patient with normal or intermittent AV conduction block and abnormal ventricular synchrony (e.g., as evidenced by a wide QRS waveform), the clinician may program a “maximize CSP” therapy to promote pacing of the conduction system for restoring more normal ventricular synchrony, even when AV conduction is normal. This CSP therapy may be referred to as “maximize CSP” because even though intrinsic AV conduction may be normal, ventricular pacing via the conduction system is promoted to occur more frequently, e.g., more often than during the minimize CSP therapy, to improve ventricular synchrony.

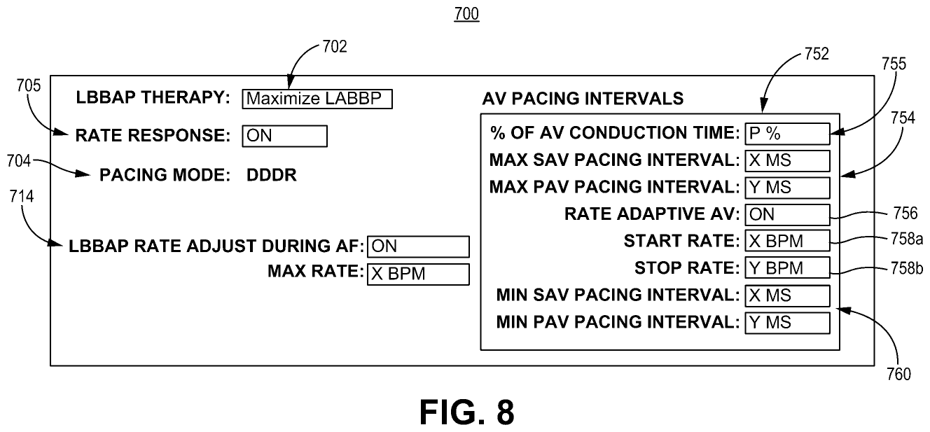

A patient having complete AV block is pacing dependent, requiring ventricular pacing for each heartbeat. In this case, the clinician may program the CSP therapy to be “complete AV block therapy” to promote ventricular pacing via the conduction system for all or at least nearly all cardiac cycles (e.g., 100% of ventricular cycles may be paced ventricular cycles at least when a ventricular tachyarrhythmia is not occurring). The patient having complete AV block may have normal or abnormal ventricular synchrony. Pacing of the ventricles via the conduction system promotes a more physiological activation of the ventricles (e.g., compared to ventricular myocardial pacing) and may avoid long term risks associated with right ventricular apical pacing. For the sake of convenience, these three CSP therapies are referred to herein as minimum CSP therapy, maximize CSP therapy and complete AV block therapy even though it is recognized that the ventricular pacing burden during complete AV block therapy can be higher (100% or nearly 100%) than during the maximize CSP therapy when sensing of conducted ventricular depolarizations could occur. As further described below, e.g., in conjunction with FIGS. 8-10, the user selection of the CSP therapy may cause external device 50 to present pacing control parameters used by pacemaker 14 specific to the selected CSP therapy. Pacemaker 14 is configured to operate according to the pacing control parameters that are specific to the selected CSP therapy in response to receiving a corresponding CSP therapy command from external device 50.

External device 50 may be embodied as a programmer used in a hospital, clinic or physician's office to program pacemaker 14 and to acquire data from pacemaker 14. External device 50 may alternatively be embodied as a handheld device. In some examples, external device 50 is a home monitor configured to interrogate pacemaker 14 to receive signals or data from pacemaker 14 and transmit data to pacemaker 14 via a wireless communication link 48.

External device 50 may include a processor 52, memory 53, display unit 54, user interface unit 56, and telemetry unit 58. Processor 52 is coupled to the other components and units of external device 50, e.g., via a data bus, for controlling the functions attributed to external device 50 herein. Processor 52 may execute instructions stored in memory 53. Processor 52 may include any one or more of a microprocessor, a controller, a digital signal processor (DSP), an application specific integrated circuit (ASIC), a field-programmable gate array (FPGA), or equivalent discrete or analog logic circuitry. In some examples, processor 52 may include multiple components, such as any combination of one or more microprocessors, one or more controllers, one or more DSPs, one or more ASICs, or one or more FPGAs, as well as other discrete or integrated logic circuitry. The functions attributed to processor 52 herein may be embodied as software, firmware, hardware or any combination thereof. Memory 53 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media.

User interface unit 56 may include a mouse, touch screen, keypad or the like to enable a user to interact with external device 50, e.g., to initiate and terminate an interrogation session for retrieving data from pacemaker 14, adjust settings of display unit 54, enter programming commands or selections or make other user requests. Display unit 54, which may include a liquid crystal display, light emitting diodes (LEDs) and/or other visual display components, may generate a display of cardiac electrical signals received from pacemaker 14 and/or data derived therefrom. Display unit 54 may be configured to generate a graphical user interface (GUI) including various windows, icons, user selectable menus, etc. to facilitate interaction by a user with the external device 50. As described below in conjunction with FIGS. 8-10, display unit 54 may display various programming windows to a user, e.g., in a GUI, for enabling programming of the CSP therapies described herein. Display unit 54 may function as an input and/or output device using technologies including liquid crystal displays (LCD), quantum dot display, dot matrix displays, light emitting diode (LED) displays, organic light-emitting diode (OLED) displays, cathode ray tube displays, e-ink, or monochrome, color, or any other type of display capable of generating tactile, audio, and/or visual output. In some examples, display unit 54 is a presence-sensitive display. Display unit 54 may serve as a user interface device that operates both as one or more input devices and one or more output devices.

In response to data received from processor 52, display unit 54 may generate a visual representation of cardiac electrical signals and settings of user programmable parameters and pacing control parameters used by pacemaker 14 based on a programmed CSP therapy as further described below. External device 50 may receive data, via telemetry unit 58, from pacemaker 14 via the wireless communication link 48. Data received from pacemaker 14 may include cardiac signals, e.g., intracardiac electrogram (EGM) signals sensed by pacemaker 14, marker channel data indicating the timing of pacing pulses delivered by pacemaker 14 and sensed cardiac event signals (e.g., sensed P-waves and sensed R-waves), data relating to the pacing history, and the currently programmed CSP therapy and control parameters.

Telemetry unit 58 includes a transceiver and antenna configured for bidirectional communication with a telemetry circuit, also referred to herein as a “communication circuit,” included in an implantable pacemaker 14, e.g., in response to user requests. Telemetry unit 58 includes communication circuitry configured to operate in conjunction with processor 52 for sending and receiving data relating to pacemaker functions via a wireless communication link 48 with the implantable pacemaker 14. Communication link 48 may be established using a radio frequency (RF) link such as BLUETOOTH®, Wi-Fi, Medical Implant Communication Service (MICS) or other communication bandwidth. In some examples, external device 50 may include a programming head that is placed proximate pacemaker 14 to establish and maintain a communication link 48, and in other examples external device 50 and pacemaker 14 may be configured to communicate using a distance telemetry algorithm and circuitry that does not require the use of a programming head and does not require user intervention to maintain a communication link 48.

It is contemplated that external device 50 may be in wired or wireless connection to a communications network via telemetry unit 58 that includes a transceiver and antenna or via a hardwired communication line for transferring data to a centralized database or computer to allow remote management of the patient. Remote patient management systems including a centralized patient database may be configured to utilize the presently disclosed techniques to enable a clinician to program a CSP therapy and corresponding programmable pacing control parameters as well as view data retrieved from pacemaker 14. Review of EGM signals, marker channel data or other data collected from pacemaker 14 may be performed remotely by a clinician who may authorize programming of a CSP therapy and pacing control parameters in pacemaker 14.

FIG. 1B is a conceptual diagram of a leadless intracardiac pacemaker 114 positioned within the RA for providing CSP in the area of the His bundle. Pacemaker 114 may be included in an IMD system configured to provide CSP according to the techniques disclosed herein. Pacemaker 114 may include a distal tip electrode 132 extending away from a distal end 112 of the pacemaker housing 115. Intracardiac pacemaker 114 is shown implanted in the RA of the patient's heart to place distal tip electrode 132 for delivering CSP pulses to the His bundle from a right atrial approach. For example, the distal tip electrode 132 may be inserted into the inferior end of the interatrial septum, beneath the AV node and near the tricuspid valve annulus to position tip electrode 132 in, along or proximate to the His bundle. In other examples, leadless intracardiac pacemaker 114 may be implanted within the right ventricle, e.g., high along the ventricular septum, for positioning distal tip electrode 132 in the vicinity of the His bundle or along the native His-Purkinje system. Distal tip electrode 132 may be a helical electrode providing fixation to anchor the pacemaker 114 at the implant position. In other examples, pacemaker 114 may include a fixation member that includes one or more tines, hooks, barbs, helices or other fixation member(s) that anchor the distal end of the pacemaker 114 at the implant site.

A proximal portion of the distal tip electrode 132 may be electrically insulated such that only the most distal end of tip electrode 132, furthest from housing distal end 112, is exposed to provide targeted pacing at a tissue site that includes a portion of the His bundle. One or more housing-based electrodes 120 and 134 may be carried on the surface of the housing 115 of pacemaker 114, proximal to distal end 112. Electrodes 120 and 134 are shown as ring electrodes circumscribing the longitudinal sidewall of pacemaker housing 115 that extends from the housing distal end 112 to housing proximal end 110. In other examples, a return anode electrode used in sensing and pacing may be positioned on housing proximal end 110. Pacing of the His-Purkinje system may be achieved using the distal tip electrode 132 as the cathode electrode and either of the housing-based electrodes 120 or 134 as the return anode. In some examples, pacing of atrial tissue may be achieved by delivering atrial pacing pulses via the distal ring electrode 120 using proximal ring electrode 134 as the return anode electrode. CSP pulses may be delivered in the area of the His bundle via tip electrode 132 with proximal ring electrode 134 as the return anode. In this way, dual chamber pacing of the atria and the ventricles (via the native conduction system) may be delivered by leadless pacemaker 114 according to the programmable CSP therapies described herein. In other examples more than two ring electrodes may be provided on housing 115, e.g., to provide two distinct atrial pacing and CSP electrode vectors.

Cardiac electrical signals produced by heart 8 may be sensed by pacemaker 114 using one or more sensing electrode pairs selected from electrodes 120, 132 and 134. For example, a ventricular electrical signal may be sensed using distal tip electrode 132 and distal electrode 120 or proximal electrode 134. Intrinsic R-waves may be sensed by the ventricular electrical signal sensing electrode pair for use in inhibiting a scheduled CSP pulse and scheduling the next CSP pulse. An atrial electrical signal may be sensed using electrodes 120 and 134, for example. Intrinsic P-waves may be sensed by the atrial electrical signal sensing electrode pair for use in inhibiting and scheduling atrial pacing pulses and/or for scheduling atrial synchronous CSP pulses. The cardiac electrical signals sensed by pacemaker 114 may be used for determining the atrial rate, ventricular rate, and/or for detecting atrial and/or ventricular tachyarrhythmias.

While external device 50 is not shown in FIG. 1B it is to be understood that pacemaker 114 can be configured to communicate via a communication circuit with external device 50 for receiving programming commands and transmitting data to external device 50 as generally described above in conjunction with FIG. 1A. Leadless pacemaker 114 may be configured to receive a CSP therapy command from external device 50 and control atrial pacing and CSP pulses according to control parameters selected by a control circuit of pacemaker 114 based on the received CSP therapy command as further described below.

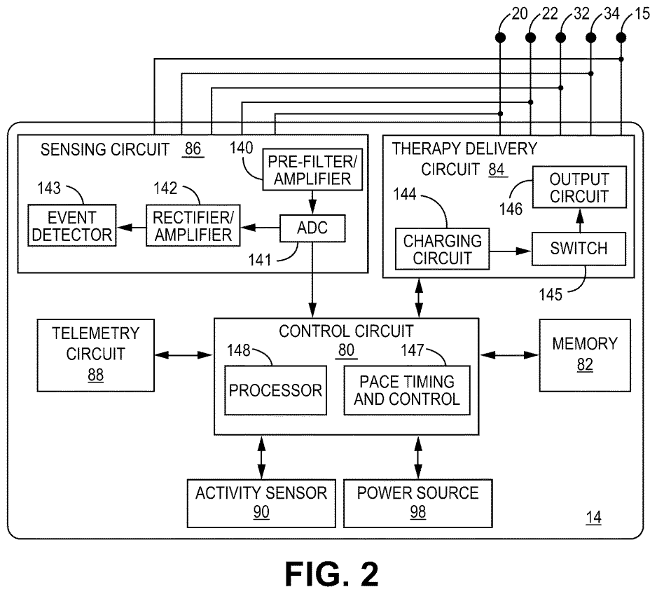

FIG. 2 is a conceptual diagram of circuitry that may be enclosed within implantable pacemaker 14 configured to deliver CSP therapies and sense cardiac electrical signals according to the techniques disclosed herein. The diagram of FIG. 2 is described with reference to pacemaker 14 connected to RA lead 16 carrying electrodes 20 and 22 and CSP lead 18 carrying electrodes 32 and 34. It is to be understood, however, that the functionality attributed to the various circuits and components shown in FIG. 2 may correspond to circuitry enclosed in a pacemaker configured to perform the techniques disclosed herein when connected to other pacing lead and electrode configurations. For instance, when pacemaker 14 is configured as an implantable cardioverter defibrillator (ICD), coil electrodes 36 and 38 may be coupled to therapy delivery circuit 84 for delivering CV/DF shocks and may be coupled to sensing circuit 86 for use in sensing cardiac electrical signals. Pacemaker 14 may be configured to receive one or more leads for electrical connection to circuitry enclosed by housing 15 to enable selection of a variety of pacing and/or sensing electrode configurations for sensing cardiac electrical signals and delivering CSP therapies. Housing 15 is depicted in FIG. 2 as an electrode coupled to pacemaker circuitry in FIG. 2, e.g., for use in a unipolar pacing and/or sensing electrode vector. In other examples, a pacemaker configured to perform the techniques disclosed herein may be a leadless pacemaker, e.g., pacemaker 114 shown in FIG. 1B, including housing-based electrodes for sensing atrial signals, delivering atrial pacing pulses, sensing ventricular signals and delivering CSP pulses. In this case, the multiple housing based electrodes, e.g., electrodes 120, 132 and 134 shown in FIG. 1B, can be coupled to sensing circuit 86 and therapy delivery circuit 84. The circuitry and functionality of pacemaker 14 described in FIG. 2 may be adapted for implementation in the leadless pacemaker 114 for performing CSP according to a programmed CSP therapy as described herein.

The electronic circuitry enclosed within housing 15 (shown conceptually as an electrode in FIG. 2) includes software, firmware and hardware that cooperatively monitor cardiac electrical signals, determine when a pacing pulse is necessary, and deliver electrical pacing pulses to the patient's heart as needed according to a programmed CSP therapy and other pacing control parameters as further described below. The electronic circuitry may include a control circuit 80, memory 82, therapy delivery circuit 84, sensing circuit 86, telemetry circuit 88 (also referred to herein as a “communication circuit”), an activity sensor 90 and power source 98.

Power source 98 provides power to the circuitry of pacemaker 14 including each of the components 80, 82, 84, 86, 88 and 90 as needed. Power source 98 may include one or more energy storage devices, such as one or more rechargeable or non-rechargeable batteries. The connections between power source 98 and each of the other components 80, 82, 84, 86, 88 and 90 are to be understood from the general block diagram of FIG. 2 but are not shown for the sake of clarity. For example, power source 98 may be coupled to one or more charging circuits included in therapy delivery circuit 84 for providing the power needed to charge holding capacitors included in therapy delivery circuit 84 that are discharged at appropriate times under the control of control circuit 80 for delivering pacing pulses. Power source 98 is also coupled to components of sensing circuit 86 (such as sense amplifiers, analog-to-digital converters, switching circuitry, etc.), telemetry circuit 88, activity sensor 90 and memory 82 to provide power to the various components and circuits as needed.

The components shown in FIG. 2 represent functionality included in pacemaker 14 (or pacemaker 114) and may include any discrete and/or integrated electronic circuit components that implement analog and/or digital circuits capable of producing the functions attributed to pacemaker 14 (and pacemaker 114) herein. The various components may include an application specific integrated circuit (ASIC), an electronic circuit, a processor (shared, dedicated, or group) and memory that execute one or more software or firmware programs, a combinational logic circuit, state machine, or other suitable components or combinations of components that provide the described functionality. Providing software, hardware, and/or firmware to accomplish the described functionality in the context of any modern medical device, given the disclosure herein, is within the abilities of one of skill in the art.

Control circuit 80 communicates, e.g., via a data bus, with therapy delivery circuit 84 and sensing circuit 86 for cooperatively sensing cardiac electrical signals and controlling delivery of cardiac electrical stimulation pulses in response to sensed cardiac event signals (or absence thereof), e.g., R-waves attendant to ventricular depolarization and/or P-waves attendant to atrial depolarization, in accordance with a programmed CSP therapy. Electrodes 20, 22, 32 and 34 and housing 15 may be electrically coupled to therapy delivery circuit 84 for delivering electrical stimulation pulses generated by therapy delivery circuit 84. Electrodes 20, 22, 32 and 34 and housing 15 may be electrically coupled to sensing circuit 86 for sensing cardiac electrical signals produced by the heart. Sensing circuit 86 may sense intrinsic signals (such as intrinsic P-waves and intrinsic R-waves) produced by the heart in the absence of a pacing pulse that captures the heart. Sensing circuit 86 may sense evoked response signals, e.g., pacing-evoked P-waves and pacing evoked R-waves, following a delivered pacing pulse of sufficient energy to cause cardiac capture.

Sensing circuit 86 may include an input pre-filter and amplifier 140 for receiving a cardiac electrical signal from a pair of sensing electrodes, e.g., RA lead electrodes 20 and 22 or CSP lead electrodes 32 and 34. The filtered and amplified signal may be passed to an analog-to-digital converter (ADC) 141 and a wide bandpass filter for producing a multi-bit digital cardiac electrical signal that may be passed to control circuit 80 and is referred to herein as a cardiac electrogram or “EGM” signal when the raw signal is sensed from electrodes on or within a heart chamber. Features of the EGM signal may be determined by processor 148 of control circuit 80 for use in detecting cardiac tachyarrhythmias in some examples.

Sensing circuit 86 may further include a rectifier and narrowband filter/amplifier 142 for receiving the ADC signal and passing a rectified, filtered signal to cardiac event detector 143. Cardiac event detector 143 may produce a cardiac sensed event signal, e.g., a sensed ventricular event (Vsense) signal or a sensed atrial event (Asense) signal, in response to the rectified signal crossing a sensing threshold amplitude, e.g., an R-wave sensing threshold amplitude or a P-wave sensing threshold amplitude, respectively. The sensed event signal is passed to control circuit 80 for use in controlling pacing pulses. For example, in response to receiving an Asense signal, pace timing and control circuit 147 included in control circuit 80 may set a pacing escape interval timer for scheduling a CSP pulse at an AV pacing interval. Control circuit 80 may inhibit an atrial pacing pulse in response to the Asense signal and schedule a subsequent atrial pacing pulse by starting an atrial lower rate interval (LRI) to provide bradycardia pacing of the atria when the LRI expires without an Asense signal being received from sensing circuit 86.

In response to receiving a Vsense signal, a scheduled CSP pulse may be inhibited and a CSP interval, e.g., a ventricular LRI, may be started for scheduling a CSP pulse. If the ventricular LRI expires before a Vsense signal is received and before an Asense signal is received or an atrial pacing pulse is delivered for triggering an atrial synchronous CSP pulse at the AV pacing interval, therapy delivery circuit 84 may deliver the scheduled CSP pulse to pace the ventricles.

Pace timing and control circuit 147 may include various timers or counters for counting down various pacing escape intervals set according to a permanent or temporary pacing mode. The pacing modes may include single chamber atrial pacing, dual chamber atrial synchronous CSP, and dual chamber atrial asynchronous CSP. A sensed event signal may cause pace timing and control circuit 147 to trigger or inhibit a pacing pulse depending on the particular pacing mode of the CSP therapy. As further described below, control circuit 80 may automatically switch between a dual chamber atrial synchronous CSP mode, e.g., a DDD pacing mode, and a dual chamber atrial asynchronous CSP mode, e.g., a DDI pacing mode, when control circuit 80 detects a fast atrial rate or atrial tachyarrhythmia based on received Asense signals and/or EGM analysis by processor 148. Depending on the programmed CSP therapy, control circuit 80 may automatically switch between a single chamber atrial pacing mode, e.g., an AAI pacing mode, and a dual chamber atrial synchronous CSP mode, e.g., a DDD pacing mode, based on an AV conduction time that may be measured during an AV conduction check, as further described below.

Sensing circuit 86 may include multiple sensing channels, e.g., a ventricular sensing channel and an atrial event sensing channel. For example, sensing circuit 86 may receive an atrial signal from atrial electrodes 20 and 22, which may be filtered, amplified, rectified and passed to an atrial event detector of event detector circuit 143. Cardiac event detector 143 may generate an Asense signal in response to the atrial signal crossing a P-wave sensing threshold. A ventricular sensing channel of sensing circuit 86 may include a different narrowband filter than the atrial sensing channel. A ventricular event detector of event detector 143 can be configured to generate a Vsense signal in response to the narrowband filtered ventricular signal crossing an R-wave sensing threshold. Each atrial sensing channel and ventricular sensing channel may include separate pre-filter/amplifiers 140, ADC 141, rectifier and narrowband filter/amplifier 142 and event detector 143 or some components may be shared between the different sensing channels. Cardiac event detector 143 is a circuit or component including one or more sense amplifiers, comparators and/or other components configured to receive the filtered and amplified atrial and ventricular signals, compare the signals to respective P-wave and R-wave sensing thresholds, and generate respective Asense and Vsense signals passed to control circuit 80.

Control circuit 80 may be configured to control therapy delivery circuit 84 to deliver the programmed CSP therapy according to an automatically selected pacing mode and programmed pacing control parameters. Therapy delivery circuit 84 is configured to generate pacing pulses and includes a charging circuit 144 including one or more charge storage devices such as one or more holding capacitors, an output circuit 146, and switching circuitry 145. Switching circuitry 145 can be controlled by control signals from control circuit 80 to control when the holding capacitor(s) of charging circuit 144 are charged and when the charged holding capacitor(s) are discharged through the output circuit 146 to deliver a pacing pulse via selected pacing electrode vector(s) for pacing the atria and/or the ventricles (via the His-Purkinje conduction system) according to the programmed CSP therapy.

Output circuit 146 may include switching circuitry for selecting the pacing electrode vector(s) and associated pacing electrode polarities coupled to a holding capacitor of charging circuit 144 via switching circuitry 145. For instance, output circuit 146 may include switching circuitry for selecting the CSP lead tip electrode 32 as a pacing cathode electrode with return anode ring electrode 34 for bipolar pacing in the area of the His bundle, LBB or RBB. Alternatively, ring electrode 34 may be selected as a cathode electrode with tip electrode 32 selected as the return anode in a bipolar pacing electrode vector for delivering CSP in the area of the LBB or the RBB. The RA lead electrodes 20 and 22 may be selected by switching circuitry included in output circuit 146 in an atrial pacing electrode vector for delivering atrial pacing pulses.

Charging of a holding capacitor to a programmed pacing voltage amplitude and discharging of the capacitor for a programmed pacing pulse width may be performed by therapy delivery circuit 84 according to control signals received from control circuit 80. For example, pace timing and control circuit 147 included in control circuit 80 may include programmable digital counters set by a microprocessor of processor 148 for controlling the basic pacing time intervals, which can also be referred to as “escape intervals,” associated with various single chamber and dual chamber pacing modes. Control circuit 80 may also set the amplitude, pulse width, polarity, or other characteristics of the cardiac pacing pulses, which may be based on programmed values stored in memory 82.

Therapy delivery circuit 84 may include multiple pacing channels for delivering pacing pulses to the RA and to the area of the LBB, RBB, His bundle or other locations along the native conduction system. Each pacing channel may be coupled to selected electrodes via switching circuitry included in output circuit 146 for selecting various unipolar or bipolar pacing electrode combinations for delivering pacing according to a programmed CSP therapy and automatically selected pacing mode as described herein. In some examples, multiple pacing channels may include an additional left ventricular pacing channel, e.g., when pacemaker 14 is coupled to a coronary sinus lead, e.g., to facilitate delivery of cardiac resynchronization pacing therapy. When pacemaker 14 is configured to deliver high voltage CV/DF shocks, therapy delivery circuit 84 may include a high voltage therapy circuit for generating high voltage CV/DF shock pulses in addition to a low voltage therapy circuit that generates cardiac pacing pulses.

Activity sensor 90 may be provided for sensing a signal correlated to patient physical activity for use by control circuit 80 in controlling rate response pacing. In one example, activity sensor 90 includes an accelerometer, e.g., a single or multi-axis piezoelectric sensor or MEMS device, for sensing an acceleration signal. Activity sensor 90 may produce an electrical signal correlated to motion or vibration of activity sensor 90 (and pacemaker 14), e.g., when subjected to patient body motion. Activity sensor 90 may include one or more filter, amplifier, rectifier, analog-to-digital converter (ADC) and/or other components for producing an acceleration signal that may be passed to control circuit 80 for use in determining a patient physical activity metric for controlling rate response pacing.

In various examples, the acceleration signal received from activity sensor 90 may be filtered by a band pass or low pass filter, e.g., a 1-10 Hz bandpass filter or a 10 Hz low pass filter, digitized by an ADC and rectified for use by processor 148 of control circuit 80 for determining a patient physical activity metric. Various activity metrics may be derived from the acceleration signal by control circuit 80 that are correlated to patient physical activity. For instance, the activity metric derived from the acceleration signal may be obtained by integrating the absolute value of an acceleration signal received from activity sensor 90 over a predetermined time duration (such as 2 seconds). The amplitude of the sampled data points over a two-second interval may be summed to obtain the activity metric. This activity metric may be referred to as an “activity count” and is correlated to the acceleration due to patient body motion imparted on the pacemaker 14 during the predetermined time interval. The 2-second (or other time interval) activity counts may be used by control circuit 80 for determining a sensor indicated pacing rate (SIR) for use in controlling rate response pacing. A patient activity metric or SIR may be used to control the rate of atrial or ventricular pacing pulses according to a temporary rate response pacing interval based on the SIR that is shortened by control circuit 80 from the LRI. A variety of methods may be used for sensing a patient activity signal and controlling rate response pacing that can be implemented in conjunction with the CSP therapy techniques disclosed herein.

Telemetry circuit 88, also referred to herein as a “communication circuit” of IMD 14 (or 114), includes a transceiver and antenna for communicating with external device 50 (shown in FIG. 1A) using radio frequency communication or other communication protocols as described above. Control parameters utilized by control circuit 80 for sensing cardiac events, analyzing EGM signals, and controlling CSP therapy may be programmed into memory 82 via telemetry circuit 88 for retrieval and execution by processor 148 of control circuit 80. Under the control of control circuit 80, telemetry circuit 88 may receive downlink telemetry from and send uplink telemetry to the external device 50.

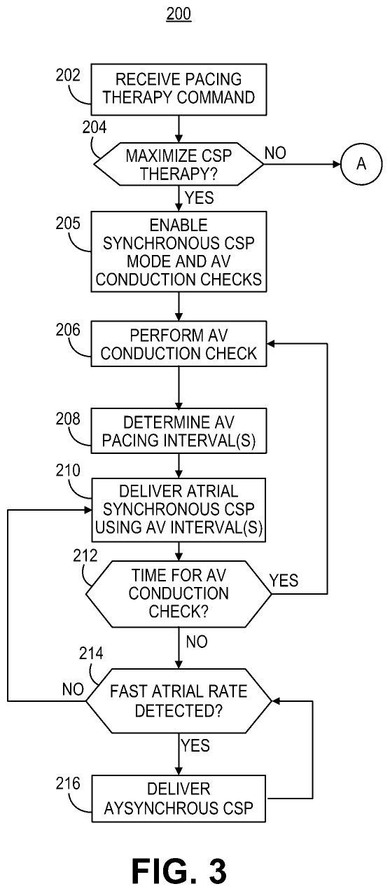

FIG. 3 is a flow chart 200 of a method performed by an IMD, such as pacemaker 14 of FIG. 1A or pacemaker 114 of FIG. 1B, for controlling CSP therapy according to some examples. With continued reference to FIG. 2, at block 202 control circuit 80 receives a CSP therapy command. The CSP therapy command can be received by telemetry circuit 88 from external device 50 and passed to control circuit 80. The CSP therapy command can be transmitted by external device 50 after a user enters a CSP therapy selection, for example, via a GUI displayed by display unit 54. The CSP therapy may be a minimize CSP therapy, a maximize CSP, or a complete AV block therapy.

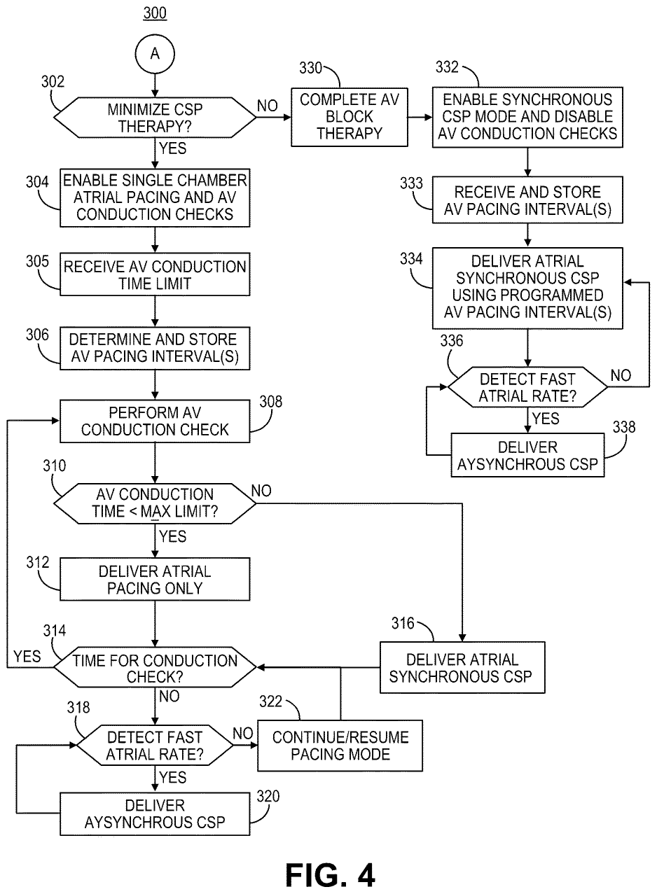

When the patient is diagnosed with a relatively wide QRS complex, indicating poor ventricular synchrony, the clinician may program the maximize CSP therapy. When the received CSP therapy command is the maximize CSP therapy command (“yes” branch of block 204), control circuit 80 enables at least an atrial synchronous CSP mode and enables AV conduction checks at block 205. Control circuit 80 may further enable switching between the atrial synchronous CSP mode and an atrial asynchronous CSP mode that can be used temporarily during fast atrial rates, e.g., a detected atrial tachyarrhythmia, as described below. Control circuit 80 may disable a single chamber atrial pacing mode that may be available when the received CSP therapy command is a minimize CSP therapy command (as described below in conjunction with FIG. 4). Even if AV conduction is good in a given patient, poor ventricular synchrony may warrant the maximize CSP therapy to promote improvement in ventricular synchrony.

Control circuit 80 may perform an AV conduction check at block 206. Control circuit 80 may perform the AV conduction check by scheduling a CSP pulse by starting a relatively long test AV pacing interval in response to an atrial event, e.g., a sensed P-wave or a delivered atrial pacing pulse. The test AV pacing interval may be set to a maximum AV pacing interval available or a specified AV conduction test value, which may be at least 200 milliseconds (ms), at least 250 ms, or at least 300 ms as non-limiting examples. In this way, therapy delivery circuit 84 is controlled to deliver a CSP pulse, e.g., via CSP lead 18, when the test AV pacing interval expires without receiving a Vsense signal from sensing circuit 86. In this case, AV block may be detected by control circuit 80 when a Vsense signal is not received during the test AV pacing interval and a CSP pulse is delivered at the expiration of the test AV pacing interval. However, when control circuit 80 receives a Vsense signal from sensing circuit 86 prior to the expiration of the AV pacing interval, the scheduled CSP pulse is inhibited by control circuit 80 (so that it is not delivered by therapy delivery circuit 84).

Control circuit 80 may determine an AV conduction time based on the time interval extending from the Asense signal or delivered atrial pacing pulse to the Vsense signal received from sensing circuit 85 during the test AV pacing interval. The Vsense signal may be sensed by sensing circuit 86 from the ventricular electrical signal sensed using tip electrode 32 and ring electrode 34 of CSP lead 18 (or tip electrode 132 and ring electrode 134 of leadless pacemaker 114). Other sensing electrode vectors may be used, however, for sensing an R-wave during a test AV pacing interval during the AV conduction check for determining an AV conduction time.

The AV conduction check may be performed at block 206 on one or more cardiac cycles. For example, control circuit 80 may start the test AV pacing interval in response to each of two, three, four or other specified number of sensed P-waves and/or atrial pacing pulses. In some examples, the AV conduction check may be performed during atrial sensing for measuring Asense to Vsense time intervals for determining a sensed AV (SAV) conduction time. Additionally or alternatively, control circuit 80 may perform the AV conduction check during atrial pacing for measuring Apace to Vsense time intervals for determining a paced AV (PAV) conduction time. The SAV conduction time and/or the PAV conduction time may be each be determined from one or multiple AV conduction check cardiac cycles, e.g., by determining the maximum, median, average or other representative value of the Asense to Vsense time intervals or Apace to Vsense time intervals, respectively, determined from multiple cardiac cycles. The SAV conduction time and the PAV conduction time are representative of the intrinsic AV conduction time following an intrinsic P-wave or following a delivered atrial pacing pulse, respectively.

At block 208, control circuit 80 determines an AV pacing interval for use in controlling CSP pulse timing during the maximize CSP therapy. Control circuit 80 may establish an AV pacing interval as a percentage of the AV conduction time determined from the AV conduction check. Control circuit 80 may establish the AV pacing interval to be between 40% and 70% of the AV conduction time, between 50% and 60% of the AV conduction time, or between 52% and 58% of the AV conduction time as examples. In one example, the AV pacing interval is set to 56% of the intrinsic AV conduction time determined from the AV conduction check. Control circuit 80 may establish two AV pacing intervals, an SAV pacing interval that is used to schedule a CSP pulse in response to receiving an Asense signal from sensing circuit 86 and a PAV pacing interval that is used to schedule a CSP pulse in response to an atrial pacing pulse being delivered by therapy delivery circuit 84.

The SAV pacing interval may be set to 56% or another specified percentage of the determined SAV conduction time. In some examples, the SAV pacing interval may be set to a specified percentage of the determined SAV conduction time up to a programmed maximum SAV interval. The clinician may program a maximum limit of the SAV pacing interval. For example, the maximum SAV pacing interval may be between 100 and 150 ms and may be 120 ms in an example.

The PAV pacing interval may be set to 56% or another specified percentage of the PAV conduction time. The clinician may program a maximum limit of the PAV pacing interval. In some examples, the PAV pacing interval may be set to a specified percentage of the determined PAV conduction time up to the programmed maximum PAV pacing interval. The maximum PAV pacing interval may be between 120 and 180 ms and may be 150 ms in an example. In some examples, the PAV pacing interval may be determined to be an offset greater than a determined SAV pacing interval (so that only the SAV conduction time needs to be determined at block 206). Alternatively, the SAV pacing interval could be determined by control circuit 80 to be an offset less than a determined PAV pacing interval (so that only the PAV pacing interval needs to be determined at block 206).

At times, control circuit 80 may detect AV block by performing the AV conduction check at block 206. AV block can be detected by control circuit 80 when no Vsense signals are received during test AV pacing intervals such that an AV conduction time cannot be determined. The patient may be experiencing AV block or an AV conduction time that is longer than the test AV pacing interval. In this case, control circuit 80 may determine the AV pacing interval at block 208 based on the programmed maximum AV pacing interval. As indicated above, the user may program a maximum limit of the SAV pacing interval and a maximum limit of the PAV pacing interval. When AV block is detected during the AV conduction check, control circuit 80 may set the SAV pacing interval to the programmed maximum SAV pacing interval and set the PAV pacing interval to the programmed maximum PAV pacing interval.

At block 210, control circuit 80 controls therapy delivery circuit 84 to deliver atrial synchronous CSP using the SAV and PAV pacing intervals determined at block 208 based on the measured SAV conduction time and/or measured PAV conduction time determined at block 206. Atrial synchronous CSP can be delivered in a dual chamber pacing mode, e.g., a DDD or DDDR pacing mode. Atrial pacing pulses can be delivered in response to the expiration of an atrial LRI without an Asense signal received from sensing circuit 86. The atrial LRI which may be a temporary rate response LRI (when rate response pacing is enabled) or a programmed base rate LRI. In response to a delivered atrial pacing pulse or an Asense signal received from sensing circuit 86, control circuit 80 schedules the atrial synchronous CSP pulse by starting the respective PAV or SAV pacing interval. Therapy delivery circuit 84 may deliver a CSP pulse in response to the PAV or SAV interval expiring when a Vsense signal is not received from sensing circuit 86 by control circuit 80 before the PAV or SAV interval expires.

In some examples, as the heart rate increases, control circuit 80 may decrease the SAV and PAV pacing intervals from the starting SAV and PAV pacing interval values determined as a percentage of the SAV and PAV conduction times. The SAV and PAV pacing intervals may be increased back to the starting values with decreases in heart rate during the atrial synchronous CSP mode. Control parameters used by control circuit 80 for controlling SAV and PAV pacing intervals with changes in heart rate (e.g., changes in atrial pacing rate and/or changes in the intrinsic atrial rate) are further described below, e.g., in conjunction with FIG. 8.

Control circuit 80 may determine that it is time to repeat the AV conduction check at block 212. Control circuit 80 may be configured to perform the AV conduction check at scheduled time intervals or scheduled time(s) of day. In some examples, control circuit 80 may start an AV conduction check timer after performing an AV conduction check at block 206. The AV conduction check timer may be set between 1 minute and 30 minutes, between 2 minutes and 20 minutes, or between 5 and 10 minutes in various examples. Control circuit 80 may determine that it is time for an AV conduction check at block 212 in response to the AV conduction check timer expiring. In some examples, an AV conduction check is performed every 3 minutes, every 5 minutes, every 8 minutes or every 10 minutes though AV conduction checks may be scheduled at other, e.g., shorter or longer, time intervals according to patient need.

AV conduction checks may be scheduled at fixed or variable time intervals. In some examples, when control circuit 80 detects AV block during an AV conduction check at block 206, control circuit 80 may increase the time interval for scheduling the next AV conduction check. For example, the AV conduction check timer may be set to a time interval that is doubled, increased by 50% or increased by a specified time interval, e.g., increased by 5 minutes, 10 minutes or 30 minutes. The AV conduction check timer may initially be set to 5 minutes in an example. The AV conduction check timer may be doubled each time AV block is detected up to a maximum of 12 hours, 24 hours, 48 hours or other specified maximum AV conduction check time interval. When AV conduction is detected, e.g., based on a Vsense signal received during the test AV pacing interval, the AV conduction check timer may be reset to its initial value, e.g., 5 minutes in this example.

When it is not time for an AV conduction check (e.g., when the AV conduction check timer is still running), control circuit 80 may continue controlling therapy delivery circuit 84 to deliver atrial synchronous CSP pulses according to the established SAV and PAV intervals determined at block 208. At any time during the atrial synchronous CSP mode, control circuit 80 may detect a fast atrial rate at block 214. The fast atrial rate may correspond to sinus tachycardia or a non-sinus atrial tachyarrhythmia, atrial fibrillation (AF) or atrial flutter (AFL). Control circuit 80 may be configured to detect an atrial rate based on PPIs determined between consecutive Asense signals and/or atrial pacing pulses.

Control circuit 80 may be configured to detect an atrial rate that is faster than an upper tracking rate limit. When the atrial rate is faster than the upper tracking rate, control circuit 80 may continue to operate in the synchronous CSP mode, e.g., DDD pacing mode, but may withhold or delay a CSP pulse so that the CSP rate is not faster than the upper tracking rate. For example, when control circuit 80 receives an Asense signal at a PPI shorter than an upper tracking rate interval, a CSP pulse may not be scheduled by control circuit 80 by not starting a the SAV pacing interval until the next Asense signal. In other examples, when the atrial rate is faster than the upper tracking rate limit, e.g., faster than an atrial tachyarrhythmia rate, control circuit 80 may switch to a non-tracking CSP mode, also referred to herein as an “atrial asynchronous CSP mode” (or “asynchronous CSP mode”) at block 216. During the asynchronous CSP mode, control circuit 80 may schedule CSP pulses based on a ventricular LRI, temporary rate response LRI, or other ventricular rate interval. Control circuit 80 may control therapy delivery circuit 84 to deliver the CSP pulses to maintain ventricular rate support but without tracking Asense signals that are occurring at an atrial rate faster than the upper tracking rate limit.

In some examples, control circuit 80 may be configured to detect atrial tachyarrhythmia according to an implemented tachyarrhythmia detection algorithm. An atrial tachyarrhythmia detection algorithm may rely on PPIs (e.g., falling in an atrial tachyarrhythmia detection interval zone) and/or atrial EGM signal analysis. The techniques disclosed herein for controlling CSP according to a programmed CSP therapy are not limited to a particular method for detecting an atrial tachyarrhythmia for causing the pacing mode switch to an asynchronous CSP mode at block 216. Examples of atrial tachyarrhythmia detection methods that may be implemented in conjunction with the CSP therapy methods disclosed herein are generally disclosed in U.S. Pat. No. 11,576,607 (Higgins, et al., filed on Feb. 28, 2020), incorporated herein by reference in its entirety.

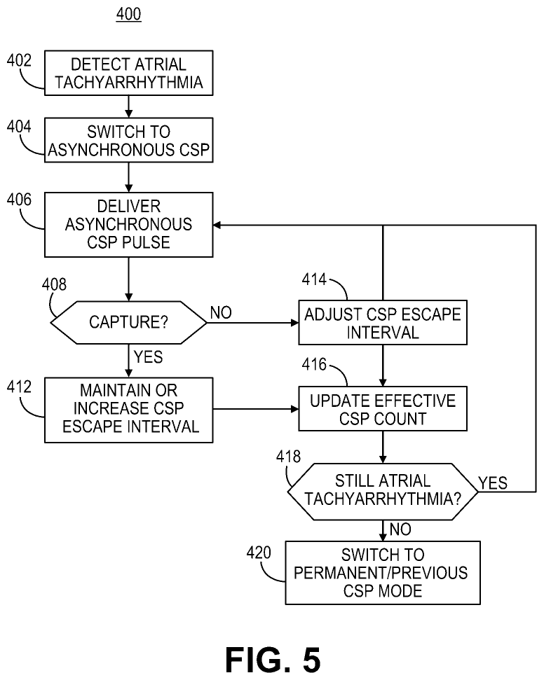

In response to detecting an atrial tachyarrhythmia at block 214, e.g., in response to detecting AF or AFL, control circuit 80 may perform a pacing mode switch from the atrial synchronous CSP to atrial asynchronous CSP at block 216. In this pacing mode, CSP pulses can be delivered in a non-tracking CSP mode such as DDI(R) pacing mode. As described below, in some examples control circuit 80 may be configured to automatically adjust the rate of asynchronous CSP pulses delivered during atrial tachyarrhythmia to promote capture by the CSP pulses for providing a regular ventricular rate. During AF, for example, rapidly conducted depolarizations may cause an irregular ventricular rate, which can be symptomatic in some patients. Methods for delivering asynchronous CSP in response to detecting an atrial tachyarrhythmia are described below, e.g., in conjunction with FIGS. 5-7.

When the atrial rate slows to less than a fast atrial rate threshold and/or atrial tachyarrhythmia is no longer being detected at block 214, control circuit 80 may switch back to the atrial synchronous CSP mode, e.g., DDD(R) pacing mode, at block 210. In some examples, control circuit 80 may return to block 206 to perform an AV conduction check in response to detecting termination of a fast atrial rate or an atrial tachyarrhythmia episode to re-establish the SAV and PAV pacing intervals. In other examples, if the AV conduction check timer is still running, control circuit 80 may return to block 210 to deliver atrial synchronous CSP using the most recent values of the SAV and PAV pacing intervals until the AV conduction check timer expires.

Referring again to block 204, control circuit 80 may receive the CSP therapy command at block 202 that is not the maximize CSP therapy command (“no” branch of block 204). When the CSP therapy command is not the maximize CSP, control circuit 80 may advance to the flow chart of FIG. 4 as indicated by connector “A.”

FIG. 4 is a flow chart 300 of a method for controlling CSP therapy delivery by an IMD, e.g., pacemaker 14 of FIG. 1A or leadless pacemaker 114 of FIG. 1B, and is a continuation of the flow chart 200 of FIG. 3. When the received CSP therapy command is not a maximize CSP therapy command (“no” branch of block 202 of FIG. 3), control circuit 80 may advance to block 302 of FIG. 4 (as shown by connector “A”). In some cases, the clinician may program the CSP therapy to be a minimize CSP therapy to promote intrinsic conduction between the atria and the ventricles. A clinician may program the minimize CSP therapy when a patient presents a normal QRS width, e.g., as observed on an ECG or the EGM retrieved from pacemaker 14/114. A normal QRS width may indicate normal conduction along the His-Purkinje conduction system and acceptable ventricular synchrony that does not necessarily require CSP. When the received pacing therapy command is the minimize CSP therapy command (“yes” branch of block 302), control circuit 80 may enable a single chamber atrial pacing mode at block 304 to provide atrial rate support as needed but promote intrinsic conduction along the AV node and native His-Purkinje system.

The patient, however, may occasionally experience AV block or a prolonged AV conduction time that can result in asystole or poor AV synchrony. In addition to enabling the single chamber atrial pacing mode at block 304, control circuit 80 may enable AV conduction checks and automatic switching between the single chamber atrial pacing mode and a dual chamber atrial synchronous CSP mode. Control circuit 80 may control therapy delivery circuit 84 to deliver single chamber atrial pacing to promote intrinsically conducted depolarizations to the ventricles during periods of time when AV conduction is intact. Control circuit 80 may control therapy delivery circuit 84 to deliver atrial synchronous CSP pulses when a long AV conduction time is measured during an AV conduction check or AV block is detected. The minimize CSP therapy may therefore include a single chamber atrial pacing mode (which may be referred to as the “primary” or “permanent” pacing mode) that can be switched to a dual chamber atrial synchronous CSP mode (which may be referred to as a “temporary pacing mode”) during periods of AV block or long AV conduction times. The minimize CSP therapy may further include a non-tracking or atrial asynchronous CSP mode that control circuit 80 may automatically switch to as a temporary pacing mode when a fast atrial rate or an atrial tachyarrhythmia is detected.

At block 305, control circuit 80 may receive a user programmed maximum AV conduction time limit. In some examples, control circuit 80 may receive a user programmed maximum SAV conduction time limit and a maximum PAV conduction time limit. The programmed maximum AV conduction time limit(s) can be stored in memory 82. In other examples, a default maximum SAV conduction time limit and default maximum PAV conduction time limit may be stored in memory 82 that are not required to be programmed by a user. As further described below, when an intrinsic SAV conduction time or an intrinsic PAV conduction time determined from an AV conduction test is greater than the respective maximum SAV conduction time limit or maximum PAV conduction time limit, control circuit 80 may automatically switch from the single chamber atrial pacing mode to a dual chamber synchronous CSP mode.

At block 306, control circuit 80 determines the AV pacing intervals to be used during the atrial synchronous CSP mode. Control circuit 80 may determine an SAV pacing interval and a PAV pacing interval at block 306. The SAV pacing interval and the PAV pacing interval may each be determined as a specified percentage of the respective maximum SAV conduction time limit and the maximum PAV conduction time limit. The specified percentage may be between 40% and 70%, between 50% and 60%, between 52% and 58% and can be 56% as examples. As such, instead of determining the SAV and PAV pacing intervals as a percentage of the measured SAV and PAV conduction times, as in the maximize CSP therapy, control circuit 80 may determine the SAV and PAV pacing intervals as a percentage of the maximum SAV conduction time limit and the maximum PAV conduction time limit. These maximum SAV and PAV conduction time limits can be programmable or stored values, rather than measured values of intrinsic AV conduction times. These maximum SAV and PAV conduction time limits are CSP control parameters used by control circuit 80 for determining when to switch between single chamber atrial pacing and dual chamber atrial synchronous CSP during the minimize CSP therapy. In other examples, the SAV and PAV pacing intervals may be programmable by a user to values that are not necessarily based on, e.g., a percentage of, the programmed maximum SAV and PAV conduction time limits. In this case, control circuit 80 may determine and store the SAV and PAV pacing intervals according to user programmed values in response to receiving the minimize CSP therapy command.

At block 308, control circuit 80 may perform an AV conduction check. The AV conduction check may be performed as generally described above by setting a test AV pacing interval to an extended or maximum available setting to allow intrinsic AV conduction to occur. In some examples, the AV conduction check can be performed by withholding ventricular pacing for one (or more) AV conduction check cycle(s). In some examples, the AV conduction check may be performed by measuring the Asense to Vsense interval(s) and/or Apace to Vsense interval(s) during the single chamber atrial pacing mode of the minimize CSP therapy. Control circuit 80 may determine an AV conduction time, which may be determined as an SAV conduction time or a PAV conduction time as generally described above in conjunction with FIG. 3.

At block 310, control circuit 80 may compare the AV conduction time(s) to the maximum AV conduction time limit. For example, the SAV conduction time may be compared to the maximum SAV conduction time limit stored in memory 82. The PAV conduction time may be compared to the maximum PAV conduction time limit stored in memory 82. If the SAV conduction time and/or the PAV conduction time is/are greater than the respective maximum SAV conduction time limit and/or maximum PAV conduction time limit, control circuit 80 may detect AV conduction block. In other examples, the AV pacing interval during the AV conduction check may be set to a maximum AV conduction time limit. When a Vsense signal is not received during the AV pacing interval, control circuit 80 may detect AV conduction block without necessarily determining an SAV conduction time or PAV conduction time.

In response to detecting the AV conduction block from the AV conduction check (“no” branch of block 310), control circuit 80 may switch the pacing mode to an atrial synchronous CSP mode at block 316. The SAV pacing interval and the PAV pacing interval determined and stored at block 306 may be used by control circuit 80 in scheduling atrial synchronous CSP pulses following Asense signals and delivered atrial pacing pulses, respectively. During the minimize CSP therapy, therefore, the SAV and PAV pacing intervals can be determined by control circuit 80 as the specified percentage of the maximum SAV and PAV conduction time limits used by control circuit 80 to control pacing mode switching between the single chamber atrial pacing mode and the dual chamber atrial synchronous CSP mode. In other examples, the SAV and PAV pacing intervals are set to specified, e.g., user programmable, values that are used by control circuit 80 to schedule CSP pulses when the maximum SAV and PAV conduction time limits are exceeded as determined during an AV conduction check. In contrast, during the maximize CSP therapy described above in conjunction with FIG. 3 above, the SAV and PAV pacing intervals are set to a specified percentage of the measured SAV and PAV conduction time intervals, respectively, that are determined during the AV conduction check.

In some examples, as heart rate increases, control circuit 80 may decrease the SAV and PAV pacing intervals from the starting SAV and PAV pacing interval values determined as a percentage of the maximum SAV and PAV conduction time limits or according to programmed SAV and PAV pacing interval values. The SAV and PAV pacing intervals may be increased back to the starting values with decreases in heart rate during the atrial synchronous CSP mode. Control parameters used by control circuit 80 for controlling SAV and PAV pacing intervals with changes in heart rate (e.g., changes in atrial pacing rate and/or changes in the sensed intrinsic atrial rate) are further described below.

At block 314, control circuit 80 may determine if it is time to perform another AV conduction check. As described above, control circuit 80 may restart an AV conduction check timer when an AV conduction check is performed to schedule the next AV conduction check. If the AV conduction check timer expires, control circuit 80 may perform an AV conduction check at block 308.

When the SAV conduction time or PAV conduction time determined during an AV conduction check, is less than or equal to the respective maximum SAV conduction time limit or PAV conduction time limit (“yes” branch of block 310), control circuit 80 may control therapy delivery circuit 84 to deliver single chamber atrial pacing at block 312. If control circuit 80 is performing the AV conduction check at block 310 while operating in the atrial synchronous CSP mode (flow from block 316 to block 314 to block 310), control circuit 80 may switch the pacing mode, e.g., from a DDD(R) pacing mode to an AAI(R) pacing mode at block 312. The AV conduction check timer may be restarted to schedule the next AV conduction check. When the AV conduction check is performed while operating in the primary or permanent AAI(R) pacing mode, control circuit 80 may remain in the single chamber atrial pacing mode as long as the intrinsic AV conduction time is less than a programmed maximum limit (“yes” branch of block 310).

At any time during the single chamber atrial pacing mode or the dual chamber atrial synchronous CSP mode, control circuit 80 may detect a fast atrial rate which may be a sinus rate that is greater than the tracking upper rate limit or an atrial tachyarrhythmia, e.g., AF, at block 318. As long as a fast atrial rate is not detected at block 318 while operating in the single chamber atrial pacing mode or the dual chamber atrial synchronous CSP mode (“no” branch of block 318), control circuit 80 may continue operating in the current pacing mode at block 322. As described above, in some instances, if the atrial rate is faster than an upper tracking rate but not faster than a mode switch rate (or an atrial tachyarrhythmia is not detected), control circuit 80 may remain in an atrial synchronous CSP mode but may withhold a CSP pulse in response to an Asense signal received at a PPI that is shorter than the upper tracking rate interval.

Control circuit 80 may switch to a non-tracking, asynchronous CSP mode at block 320, such as a DDI(R) pacing mode (or VDI(R) pacing mode), in response to detecting a fast atrial rate, e.g., in response to detecting AF or AFL or an atrial rate that is faster than a mode switch rate. Example methods for delivering the asynchronous CSP during a detected fast atrial rate or atrial tachyarrhythmia are described below in conjunction with FIGS. 5-7. Therapy delivery circuit 84 may deliver CSP pulses at ventricular LRIs, which may be adjustable according to the methods described below in conjunction with FIGS. 5-7, until the fast atrial rate or atrial tachyarrhythmia is no longer detected at block 318. Control circuit 80 may switch the pacing mode from the asynchronous CSP mode, e.g., a DDI(R) pacing mode) back to the most recent pacing mode (e.g., the AAI(R) or DDD(R) pacing mode) at block 322, e.g., to resume single chamber atrial pacing or resume dual chamber atrial synchronous CSP, whichever was in effect at the time of the fast atrial rate detection.

Referring again to block 302, if the CSP therapy command is neither the maximize CSP therapy nor the minimize CSP therapy, control circuit 80 can receive a complete AV block therapy command at block 330. Control circuit 80 may enable an atrial synchronous CSP mode, e.g., a DDD(R) pacing mode at block 332 that will be in effect as the primary or permanent pacing mode as long as the complete AV block therapy is the selected therapy. The single chamber atrial pacing mode can be disabled at block 332 in response to receiving the complete AV block therapy command. Control circuit 80 may disable AV conduction checks at block 332 in response to the complete AV block therapy command.

Control circuit 80 may disable automatic pacing mode switching between a single chamber atrial pacing mode and the atrial synchronous CSP mode in response to the complete AV block CSP therapy command. Because AV conduction times are not being measured, control circuit 80 may apply SAV and PAV pacing intervals that are stored in memory 82 for controlling the timing of the atrial synchronous CSP pulses. The SAV and PAV pacing intervals may be user programmed values received at block 333 for controlling therapy delivery circuit 84 in delivering CSP pulses. Therapy delivery circuit 84 may deliver the atrial synchronous CSP pulses at block 334 scheduled at the programmed values of the SAV and PAV pacing intervals. As generally described above, during the atrial synchronous CSP mode, if a detected atrial rate is faster than an upper tracking rate, control circuit 80 may withhold or delay a CSP pulse to avoid pacing the ventricles at a rate that is faster than the upper tracking rate.

It is noted that in any of the CSP therapies, control circuit 80 may decrease the SAV and PAV pacing intervals from the starting SAV and PAV pacing interval values as the heart rate increases. During the complete AV block therapy, control circuit 80 may decrease the SAV and PAV pacing intervals from the programmed values with increasing heart rate and increase the SAV and PAV pacing interval back to the starting values with decreases in heart rate during the atrial synchronous CSP mode at block 334. In some examples, control circuit 80 adjusts the SAV and PAV pacing intervals linearly with the atrial rate, sensed or paced, between the starting value and a minimum limit of the respective SAV pacing interval or PAV pacing interval. Control parameters that may be used by control circuit 80 for controlling SAV and PAV pacing intervals with changes in heart rate are further described below, e.g., in conjunction with FIG. 8.

Automatic pacing mode switching from the atrial synchronous CSP mode to an asynchronous CSP mode may be enabled when the complete AV block therapy command is received. Control circuit 80 may detect a fast atrial rate at block 336, e.g., based on atrial tachyarrhythmia detection intervals and/or other atrial tachyarrhythmia detection criteria. When a fast atrial rate is detected at block 336, control circuit 80 may switch from the primary or permanent atrial synchronous CSP mode, e.g., a DDD(R) pacing mode, to a non-atrial tracking (asynchronous) CSP mode at block 338. The asynchronous CSP mode, e.g., a DDI(R) pacing mode, may be in effect for controlling therapy delivery circuit 84 in delivering atrial and CSP pulses until the fast atrial rate is no longer detected at block 336 (e.g., the atrial rate falls below a fast atrial rate threshold and/or an atrial tachyarrhythmia is no longer detected). Control circuit 80 may switch back to the atrial synchronous CSP mode at block 334 when the fast atrial rate or atrial tachyarrhythmia is no longer detected.

FIG. 5 is a flow chart 400 of a method for delivering CSP during a non-atrial tracking, asynchronous CSP mode in response to detecting a fast atrial rate or an atrial tachyarrhythmia. As indicated above, during any of the programmed CSP therapies, automatic pacing mode switching from the atrial synchronous CSP mode to an asynchronous CSP mode in response to detecting an atrial tachyarrhythmia, e.g., AF, may be enabled. Control circuit 80 may detect atrial tachyarrhythmia at block 402, which may generally correspond to block 214 of FIG. 3, block 318 of FIG. 4 or block 336 of FIG. 4. Control circuit 80 may detect a fast atrial rate at block 402 based on PPIs that are shorter than a fast atrial rate threshold interval, e.g., shorter than an atrial tachyarrhythmia detection interval. An atrial tachyarrhythmia detection interval may be programmable and stored in memory 82 and may be between 300 and 400 ms, between 320 and 380 ms, and can be 350 ms (or about 170 beats per minute) as examples. Control circuit 80 may detect atrial tachyarrhythmia in response to a threshold number of atrial tachyarrhythmia detection intervals, e.g., 75%, 80% or 100% of the most recent 16, 20, 24, 30, 40 or other specified number of PPIs. In other examples, additional or alternative criteria or algorithms may be applied to the cardiac electrical signals sensed by pacemaker 14/114 for detecting atrial tachyarrhythmia, e.g., based on P-wave morphology, RRI variability, or other atrial tachyarrhythmia evidence, and causing control circuit 80 to automatically switch to the non-tracking asynchronous CSP mode at block 404.

At block 406, therapy delivery circuit 84 delivers a CSP pulse upon expiration of a CSP escape interval (e.g., set by pace timing and control circuit 147 of FIG. 2). The CSP escape interval may be set to a lower rate interval (LRI) corresponding to a programmed CSP lower rate. In some examples, control circuit 80 may control the asynchronous CSP to begin at a rate smoothing interval to avoid a sudden change in ventricular rate. The rate smoothing interval may be determined by control circuit 80 based on one or more of the most recent RRIs. The RRI(s) may be determined between consecutive Vsense signal(s) and/or ventricular pacing pulse(s). For example, the starting CSP escape interval may be set equal to the most recent RRI, a median of the most recent Y RRIs, where Y may be 2 to 20 RRIs as examples.

At block 408, control circuit 80 determines if the asynchronous CSP pulse captures the ventricles. The CSP pulse is generally delivered to capture at least a portion of the His-Purkinje conduction system, e.g., the LBB, RBB, His bundle, or another portion of the His-Purkinje conduction system. The CSP pulse may capture a portion of the ventricular myocardium in addition to capturing a portion of the native conduction system. It is further recognized that in some instances the CSP pulse may fall below a conduction system capture threshold such that ventricular myocardium is captured without capturing at least a portion of the conduction system. CSP pulses, however, may be adjusted (e.g., by increasing the CSP pulse amplitude and/or pulse width) to promote a high likelihood of capturing at least a portion of the conduction system, which may or may not include capturing local ventricular myocardium. A capture test or capture threshold search may be performed by control circuit 80 to promote CSP pulse energy sufficient to capture at least a portion of the conduction system and cause a pacing evoked ventricular depolarization.

At block 408, control circuit 80 may determine if a pacing evoked response is detected after the delivered CSP pulse, indicating ventricular capture. A pacing evoked response may be detected based on a polarity of the cardiac electrical signal sensed using a unipolar sensing electrode vector. For example, the unipolar sensing electrode vector for detecting capture following a CSP pulse may include the CSP cathode electrode, e.g., tip electrode 32, and the pacemaker housing 15 or a coil electrode, e.g., coil electrode 36 or 38 (see FIG. 1A). In the case of the leadless pacemaker 114 of FIG. 1B, the sensing electrode vector for detecting an expected evoked response waveform may include tip electrode 132 and ring electrode 134. When capture occurs at the CSP cathode electrode, e.g., tip electrode 32/132, the pacing evoked depolarization may be expected to propagate away from the cathode electrode. As such, a negative polarity of the evoked response waveform in the unipolar cardiac electrical signal sensed using the CSP cathode electrode can be detected as an indication of capture at block 408. The techniques disclosed herein are not limited to a particular method for detecting ventricular capture following a CSP pulse. Various methods may be used for detecting a pacing evoked waveform morphology following a delivered CSP pulse. For instance, methods for detecting capture by a CSP pulse may include determining relative timing of maximum and/or minimum peak amplitudes of the post-pace waveform. Methods for detecting capture by a CSP pulse may generally be adapted from or employ some aspects of the effective capture tests generally disclosed in U.S. Pat. No. 9,061,157 (Ghosh, et al., filed Feb. 28, 2014), incorporated herein by reference in its entirety.

When capture is detected at block 408, control circuit 80 may maintain the CSP escape interval at its current setting at block 412. In some instances, control circuit 80 may increase the CSP escape interval so that the CSP rate can be lowered to a rate that still results in effective capture of the ventricles. The CSP rate may be held at the current rate or slowed to a slower rate, as long as the CSP pulses are determined to be effective in capturing the ventricles. In some examples, a count of the number of effective CSP pulses (e.g., CSP pulses that capture the ventricles) can be updated at block 416. Control circuit 80 may track the number of delivered CSP pulses for which capture is detected out of the total number of CSP pulses delivered during a specified time period, e.g., a 24-hour time period, one week or since implant or the last interrogation session. The percentage of CSP pulses that are known to capture may be tracked as the percentage of “effective” CSP pulses. The percentage of effective CSP pulses can be transmitted to external device 50 during the next interrogation or programming session.

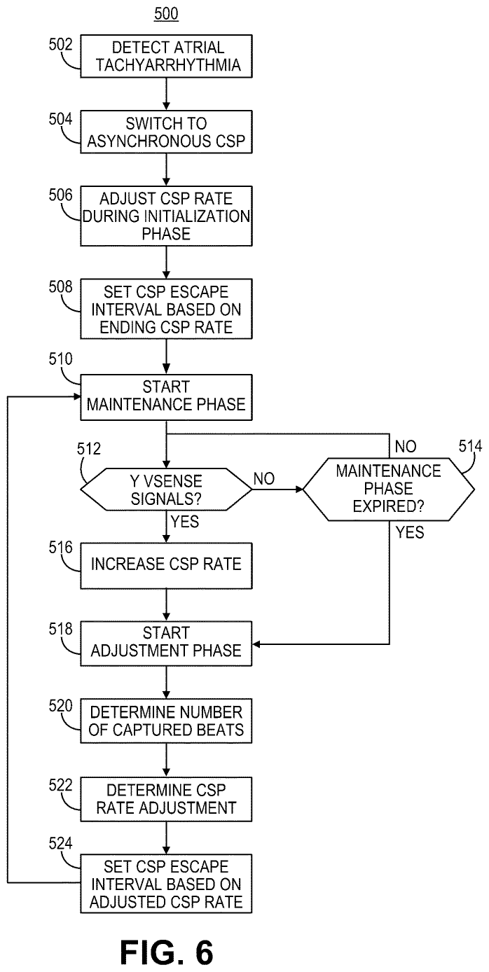

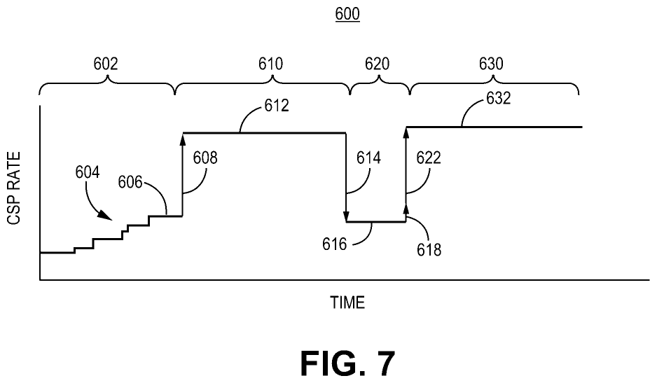

If capture is not detected at block 408, control circuit 80 may adjust the CSP escape interval at block 414. The CSP escape interval may be adjusted to decrease the CSP escape interval. In some examples, the size of the decrease in CSP interval is larger or smaller based on the current CSP escape interval length. For example, if the CSP escape interval length is relatively long, e.g., greater than 700 ms, greater than 750 ms or greater than 800 ms as examples, control circuit 80 may increase the pacing rate by shortening the CSP escape interval if capture is not detected. If the CSP escape interval length is relatively short, e.g., less than 500 ms or less than 600 ms as examples, the CSP escape interval may be increased to decrease the pacing rate if capture is not detected. When the CSP escape interval is between a long threshold interval and a short threshold interval, the CSP escape interval may be increased or decreased, e.g., depending on recent capture or non-capture detections and/or preceding CSP rate adjustments. The action taken by control circuit 80 may depend on how many out of a specified number of most recent CSP pulses were determined to capture the ventricles. Examples of CSP rate adjustments that may be made by control circuit 80 for controlling the timing of asynchronous CSP pulses during a detected atrial tachyarrhythmia are described below, e.g., in conjunction with TABLE I and FIGS. 6 and 7. As described above, control circuit 80 may optionally update a total count of delivered CSP pulses at block 416 when capture is not detected for tracking and reporting the percentage of CSP pulses that are determined to capture the ventricles as effective CSP pulses.

As long as control circuit 80 is detecting an atrial tachyarrhythmia (block 418), control circuit 80 may schedule the next asynchronous CSP pulse at block 406 by restarting the CSP escape interval, which may be adjusted in response to not detecting capture, for scheduling the next asynchronous CSP pulse at block 406. While not shown explicitly in FIG. 5, it is noted that if a Vsense signal is received during the asynchronous CSP escape interval the scheduled CSP pulse may be inhibited, and the CSP escape interval restarted. The CSP escape interval may be decreased in response to a Vsense signal to increase the CSP rate and thereby promote ventricular rate control by CSP as opposed to the conducted atrial tachyarrhythmia controlling the ventricular rate. When the atrial tachyarrhythmia is no longer detected at block 418, control circuit 80 may switch back to the permanent or primary pacing mode or the most recent pacing mode that was in effect prior to switching to the asynchronous CSP mode when the fast atrial rate was detected.

For instance, if the CSP therapy is programmed to be the minimize CSP therapy, control circuit 80 may switch to the single chamber atrial pacing mode at block 420. In some instances, if the CSP therapy is programmed to be minimize CSP therapy and the atrial synchronous CSP mode was in effect at the time that the atrial tachyarrhythmia was detected, control circuit 80 may switch back to the atrial synchronous CSP mode at block 420 when the atrial tachyarrhythmia is no longer detected. When the CSP therapy is maximize CSP therapy or complete AV block therapy, control circuit 80 may switch back to the atrial synchronous CSP mode at block 420.

FIG. 6 is a flow chart 500 of a method for performing asynchronous CSP during a fast atrial rate or an atrial tachyarrhythmia according to another example. At block 502, control circuit 80 may detect an atrial tachyarrhythmia according to any of the example methods described above. Upon detection of the atrial tachyarrhythmia, control circuit 80 may switch to the asynchronous CSP mode at block 504. The asynchronous CSP mode may be a dual chamber pacing mode, e.g., a DDI(R) pacing mode in that atrial pacing may be delivered in the absence of a sensed P-wave. In other examples, the asynchronous CSP mode may provide CSP only without atrial pacing, e.g., a VDI(R) pacing mode, due to the presence of the fast atrial rate or atrial tachyarrhythmia.

At block 506, control circuit 80 may start an initialization phase of the asynchronous CSP. A CSP pulse may be scheduled by starting a CSP escape interval set according to a starting pacing rate. The starting pacing rate may be a programmed lower rate, e.g., 40 to 60 bpm. In some examples, the starting CSP escape interval may be set to a rate smoothing interval based on one or more of the most recent RRIs (which may include Vsense to Vsense intervals, Vsense to Vpace intervals, Vpace to Vsense intervals and/or Vpace to Vpace intervals) to avoid an abrupt change in ventricular rate.

During the initialization phase, control circuit 80 may determine if CSP pulses capture the ventricles. Methods for detecting capture are described above. Based on whether a CSP pulse captures the heart or not, control circuit 80 may adjust the CSP rate by adjusting the CSP escape interval. Control circuit 80 may shorten the CSP escape interval each time capture is not detected to increase the CSP rate. If a Vsense signal is received from sensing circuit 86, control circuit 80 may shorten the CSP escape interval to increase the CSP rate. If capture is detected following delivery of a CSP pulse, the CSP escape interval may remain at its current value for scheduling the next CSP pulse. The initialization phase may be a specified time interval or number of ventricular cycles, which may include CSP cycles and Vsense cycles. The initialization phase may be 10 to 60 ventricular beats long and is 30 ventricular beats long in some examples. By the end of the initialization phase, the CSP rate is expected to be at a rate that results in a high likelihood of the CSP pulses capturing the ventricles to effectively cause ventricular depolarization and ventricular systole. The CSP rate at the end of the initialization phase may be at the minimum CSP rate that promotes ventricular capture and results in ventricular rate control by the CSP pulses (instead of conducted atrial depolarizations).

At the end of the initialization phase, control circuit 80 may set a CSP escape interval at block 508 based on the ending CSP rate (and corresponding ending CSP escape interval) reached at the end of the initialization phase. The CSP escape interval may be set equal to the initialization phase ending CSP escape interval less an offset (shorter than the ending CSP escape interval at the end of the initialization phase). The offset may be 20 to 80 ms or 40 to 60 ms as examples. The offset may be between 45 ms and 55 ms and can be 50 ms in an example. For instance, control circuit 80 may subtract 50 ms from the last CSP escape interval set at the end of the initialization phase. Control circuit 80 may start a maintenance phase of the asynchronous CSP mode at block 510 by scheduling the next CSP pulse by starting the CSP escape interval determined at block 508 (as the initialization phase ending CSP escape interval less the offset).

The maintenance phase CSP escape interval may be shortened by the offset to promote pacing evoked depolarization of the conduction system that is subsequently conducted to the ventricular myocardium. Effective capture of the conduction system may require pacing earlier than a boundary between effective capture as evidenced by the post-pace evoked response waveform and ineffective capture because of the time required for a depolarization of the conduction system to travel to and depolarize the ventricular myocardium, e.g., from the time from His Bundle, LBB or RBB depolarization to the subsequent ventricular myocardial depolarization.

In some examples, the offset may be a programmed or selected default value, e.g., 50 ms. In other examples, control circuit 80 may determine the offset based on a measured time interval from a CSP pulse (delivered at a very short AV pacing interval or a fast CSP rate) to the QRS waveform sensed from a unipolar or relatively far field cardiac electrical signal (e.g., sensed from the RA lead ring electrode 22 to the housing 15 or from the RV coil electrode 36 to the housing 15). This measured time interval can be representative of the latency of the relatively global ventricular myocardial depolarization following a CSP pulse. The offset may be determined by control circuit 80 to be equal to or a percentage of (e.g., greater than) this measured time interval from a CSP pulse to a fiducial point of the QRS waveform to provide early enough CSP pulse delivery to promote effective ventricular capture during AF that includes pacing-evoked myocardial depolarization subsequent to the CSP pulse.

In other examples, the offset may be determined as the difference between the AV pacing interval that results in optimal fusion of the CSP evoked myocardial depolarization within an intrinsically conducted ventricular depolarization and the longest AV pacing interval that still results in effective capture. Effective capture can be detected from analysis of the cardiac electrical signal sensed from the CSP tip electrode 32 to housing 15 or from the CSP tip electrode 32 to coil electrode 36, for example, as shown in FIG. 1A (or CSP tip electrode 132 to housing ring electrode 134 as shown in FIG. 1B). The AV pacing interval that results in optimal fusion of the pacing-evoked depolarization and the intrinsic ventricular depolarization may be determined by delivering CSP pulses at multiple different AV pacing intervals and analyzing the sensed cardiac electrical signal. For example, the shortest AV pacing interval used to schedule a LBBAP pulse that results in fusion of a left ventricular depolarization with the right ventricular depolarization that is intrinsically conducted from the atria may be determined as the optimal AV pacing interval for achieving fusion of the pacing-evoked and intrinsically conducted ventricular myocardial depolarizations. Fusion may be detected based on a relatively narrow QRS complex, for example. Control circuit 80 may determine the offset based on the difference between the longest AV pacing interval that still results in effective capture by the CSP pulse and the AV pacing interval that is determined to achieve fusion of the ventricular myocardial depolarization in a given patient. The determined offset may be used to shorten the ending initialization phase CSP escape interval to the maintenance phase CSP escape interval. Methods for determining an offset between an ending initialization phase CSP escape interval and a maintenance phase CSP escape interval may generally correspond to techniques for determining an advanced pacing delay disclosed in U.S. Patent Application No. 63/393,072 (Stadler, et al, filed Jul. 28, 2022), the content of which is incorporated herein by reference in its entirety.

At block 510, control circuit 80 controls therapy delivery circuit 84 to deliver CSP pulses at the pacing rate corresponding to the determined maintenance phase CSP escape interval. Therapy delivery circuit 84 may deliver CSP pulses at the CSP rate corresponding to the CSP escape interval until the maintenance phase expires at block 514. The maintenance phase may expire after a specified time interval or specified number of paced ventricular beats. For example, the maintenance interval may be between 20 seconds and 2 minutes and can be 30 seconds as examples. When the maintenance phase expires, control circuit 80 may start an adjustment phase at block 518 as described below.

In some instances, however, even though the CSP rate during the maintenance phase is selected to promote CSP with capture on every cardiac cycle, a Vsense signal may be received during a CSP escape interval. The Vsense signal can cause control circuit 80 to inhibit a scheduled CSP pulse and restart the CSP escape interval. In some examples, if a threshold number of Vsense signals are received during the maintenance phase (as determined at block 512), causing inhibition of CSP pulse delivery, control circuit 80 may increase the CSP rate at block 516 and start the adjustment phase at block 518. The maintenance phase may be terminated early in response to a threshold number of Vsense signals received from sensing circuit 86 so that control circuit 80 may “re-tune” the CSP escape interval to promote CSP pulse delivery and ventricular capture.

The threshold number of Vsense signals may be one, two or more Vsense signals. The threshold number of Vsense signals may or may not be required to be consecutive. In one example, when at least 4 out of the most recent 10 scheduled CSP are inhibited due to a Vsense signal, e.g., 4 out of 10 most recent ventricular beats are Vsense beats, control circuit 80 may increase the CSP rate at block 516, terminate the maintenance phase and start the adjustment phase at block 518. Control circuit 80 may increase the CSP rate by 1 to 5 bpm at block 516 and may increase the CSP rate by 2 bpm in an example. The CSP rate is increased by decreasing the current CSP escape interval by an appropriate decrement to increase the CSP rate by 2 bpm, for instance. If the threshold number of Vsense signals are not reached during the maintenance phase (“no” branch of block 512) and the maintenance phase expires (“yes” branch of block 514), control circuit 80 may start an adjustment phase at block 518.

When control circuit 80 starts the adjustment phase, control circuit 80 may decrease the current CSP rate by increasing the CSP escape interval currently in effect by an offset. The offset may be the same or different than the offset used at block 508 to determine the CSP escape interval at the end of the initialization phase. For example, when 50 ms is subtracted from the CSP escape interval reached at the end of the initialization phase to set the maintenance phase CSP escape interval and corresponding maintenance phase CSP rate, the offset of 50 ms may be added to the CSP interval in effect at the end of the maintenance phase, which may, in some instances, be decreased from the starting maintenance phase CSP escape interval at block 516 due to Vsense signals. Control circuit adds the offset to the ending CSP escape interval of the maintenance phase to obtain the CSP rate that is applied during the adjustment phase.

At block 518, control circuit 80 may control therapy delivery circuit 84 to deliver a specified number of CSP pulses at the decreased adjustment phase CSP rate. For example, the adjustment phase may be 3 to 20 ventricular beats long and is 5 beats long in an example. Therapy delivery circuit 84 may deliver five CSP pulses at the adjustment phase CSP escape interval set at block 516. Control circuit 80 may determine whether each delivered CSP pulse captures the ventricles or not. In some instances a Vsense signal may be received during the adjustment phase. The Vsense signal may be counted as no capture for one of the five beats (or other specified number of beats) of the adjustment phase. In response to a Vsense signal, the adjustment phase CSP escape interval may be decreased by a predetermined decrement, e.g., 10 to 20 ms, or decreased to increase the CSP rate by 2 bpm, 3 bpm or other specified rate increase to promote delivery of the CSP pulses without inhibition due to Vsense signals during the adjustment phase.

When five or another specified number of CSP pulses have been delivered and/or Vsense signals have been received during the adjustment phase, control circuit 80 may determine the number of capture detections made at block 520. Based on the number of capture detections out of the total number of ventricular beats during the adjustment phase, control circuit 80 may determine an adjustment to the CSP rate at block 522. The adjustment to the CSP rate may further depend on the current CSP rate or the associated ending maintenance phase CSP escape interval duration. As shown in the illustrative examples in TABLE I below, control circuit 80 may determine the number of delivered CSP pulses that resulted in ventricular capture and determine a CSP rate increment, decrement or no rate change based on the number of capture detections and based on the current CSP escape interval applied during the adjustment phase.

In the example of TABLE I, 5 CSP pulses may be delivered at the adjustment phase CSP escape interval. It is noted that a Vsense may be counted as a non-capture for one of the N ventricular beats of the adjustment phase. The rate adjustment determined by control circuit 80 based on the number of capture detections may be an increase of 1, 2 or 3 bpm, a decrease of 1, 2 or 3 bpm or no change. A look up table of rate adjustments, e.g., corresponding to TABLE I, may be stored in memory 82 so that control circuit 80 can retrieve the rate adjustment for a given number of capture detections (as shown in leftmost column) and a corresponding maintenance phase CSP escape interval range that represents the current effective maintenance phase CSP rate prior to starting the adjustment phase. For instance, if capture is detected for four out of five CSP pulses and the ending maintenance phase CSP escape interval is in the range of 600 ms through 750 ms, control circuit 80 may determine that the rate adjustment is a decrease of 2 bpm. In another example, if capture is detected for two out of the five delivered CSP pulses and the current CSP escape interval is less than 600 ms, the rate adjustment is zero or no change. In yet another example, if none of the CSP pulses capture the ventricles during the adjustment phase, and the current maintenance phase CSP escape interval is greater than 750 ms, control circuit 80 may determine that the rate adjustment is an increase of 3 bpm.

TABLE I represents one non-limiting example of CSP rate adjustments that may be made based on the number of CSP pulses that capture the ventricles during the adjustment phase and the currently applied CSP escape interval duration (at the end of the maintenance phase). It is contemplated that numerous variations of rate adjustments could be conceived for varying numbers or percentages of capture detections out of a total number of CSP pulses delivered during the adjustment phase. Furthermore the rate adjustments may be based on more than three CSP escape interval ranges or less than three CSP escape interval ranges and/or different numerical ranges of the CSP escape intervals may be defined than the example ranges shown in TABLE I.

TABLE I
Example values of rate adjustments that can be made
during an adjustment phase of an asynchronous CSP
mode applied during atrial tachyarrhythmia.

| | |
|---|---|
| | Rate (BPM) Increase or Decrease |
| # of capture | CSP escape interval (ms) |

| beats (out of 5) | >750 | 600 to ≤750 | <600 |
|---|---|---|---|
| 5 | −2 | −3 | −3 |
| 4 | 0 | −2 | −2 |
| 3 | 1 | 0 | −1 |
| 2 | 1 | 1 | 0 |
| 1 | 2 | 2 | 1 |
| 0 | 3 | 3 | 2 |

After determining the rate adjustment at block 522, at block 524 control circuit 80 may set the CSP escape interval to be used in the next maintenance phase based on the rate adjustment. For example, control circuit 80 may adjust the CSP rate corresponding to the pacing escape interval applied during the adjustment phase by the rate adjustment. Control circuit 80 may subtract the offset (e.g., 50 ms) from the adjusted CSP escape interval corresponding to the adjusted CSP rate. In another example, control circuit 80 may adjust the ending maintenance phase CSP escape interval by the rate adjustment for determining the starting CSP escape interval of the next maintenance phase at block 524. In this way, CSP can be delivered during the next maintenance phase according to any rate adjustments needed, as determined during the adjustment phase, to promote the delivery of CSP pulses and ventricular capture at a regular rate.

Control circuit 80 may return to block 510 to start the next maintenance phase during which therapy delivery circuit 84 delivers CSP pulses scheduled at the new updated maintenance phase CSP escape interval determined at block 524. Other examples and aspects of pacing rate adjustment methods that can be performed during atrial fibrillation are generally disclosed in U.S. Pat. No. 10,065,043 (Lu et al., filed Jun. 9, 2017) and in U.S. Pat. No. 10,213,606 (Lu et al., filed Mar. 14, 2014), both of which are incorporated herein by reference in their entirety.

FIG. 7 is a diagram 600 of a method for delivering asynchronous CSP during a detected atrial tachyarrhythmia to promote CSP and capture of the ventricles at a regular ventricular rate. Upon detecting an atrial tachyarrhythmia, control circuit 80 may start an initialization phase 602. In this example, the initialization phase may be 30 ventricular beats but could be 20 to 100 ventricular beats in other examples. The asynchronous CSP may start at a pacing rate equal to programmed lower rate, e.g., 50 bpm, 60 bpm or 70 bpm. In this example, the CSP rate is seen to be gradually increased (step increases indicated by arrow 604) by control circuit 80 during the initialization phase 602. As described above, in response to a CSP pulse that does not capture the ventricles, control circuit 80 may increase the CSP rate by 1 bpm, 2 bpm or 3 bpm as examples. By the end of the initialization phase 602, the ending CSP rate 606 is reached, which may correspond to the minimum CSP rate at which Vsense signals are not being received (resulting in no further increases in CSP rate).

At the end of the initialization phase 602, control circuit 80 increases the CSP rate from the ending initialization phase CSP rate 606 to a maintenance phase CSP rate 612. Control circuit 80 may set the maintenance phase CSP rate 612 based on the ending rate 606 of the initialization phase. For example, the initialization phase ending CSP escape interval, corresponding to the initialization phase ending CSP rate 606, may be shortened by a specified offset, e.g., 30 to 80 ms or 50 ms in the examples given above, to determine the maintenance phase CSP escape interval and corresponding maintenance phase CSP rate 612. Because the ending initialization phase CSP rate 606 may represent the minimum CSP rate at which Vsense signals are not being received, control circuit 80 may shorten the CSP escape interval by the offset to provide a CSP rate 612 slightly above the minimum CSP rate at which Vsense signals are not being received to promote CSP resulting in capture of the ventricles at a regular rate. As generally described above, the offset 608 may account for the time from a CSP pulse that depolarizes at least a portion of the conduction system to the resulting, subsequent ventricular myocardial depolarization.

Control circuit 80 controls therapy delivery circuit 84 to deliver CSP during the maintenance phase 610 at the maintenance phase CSP rate 612. The CSP may be delivered in the asynchronous CSP mode at the rate 612 until the expiration of the maintenance phase 610 as shown in FIG. 7. The maintenance phase 610 may be a specified time duration, e.g., 30 seconds, 60 seconds, or two minutes as examples. In other examples, the maintenance phase may be a specified number of CSP pulses, e.g., 30, 60, or 100 CSP pulses. While not shown in the example of FIG. 7, it is to be understood that if a threshold number of Vsense signals are received from sensing circuit 86 during the maintenance phase 610, control circuit 80 may terminate the maintenance phase 610 early and start the adjustment phase 620. As described above in conjunction with FIG. 6, control circuit 80 may increase the CSP rate 612 in response to the Vsense signals such that a CSP rate at the early termination of the maintenance phase 620 may be higher, e.g., 2 bpm higher, than the CSP rate 612 applied at the start the maintenance phase 610.

To start the adjustment phase 620, control circuit 80 may decrease the ending maintenance phase CSP rate 612 (which may be an increased rate due to Vsense signals in some instances). The ending maintenance phase CSP rate 612 may be decreased to the adjustment phase CSP rate 616 by adding an escape interval offset to the CSP escape interval corresponding to the ending maintenance phase CSP rate 612. The offset corresponding to the CSP rate decrease 614 may be 30 to 80 ms and is 50 ms in some examples. The CSP escape interval offset used by control circuit 80 to perform the rate increase 608 between the initialization phase ending rate 606 and the maintenance phase starting rate 612 may have the same magnitude as the CSP escape interval offset used by control circuit 80 to perform the rate decrease 614 between the ending maintenance phase CSP rate 612 and the adjustment phase CSP rate 616.

As described above in conjunction with FIG. 6, therapy delivery circuit 84 may deliver a specified number of CSP pulses at the adjustment phase CSP rate. The number of adjustment phase CSP pulses may be 3 to 20 pacing pulses and can be 5 pacing pulses as examples. In the example of five CSP pulses being delivered during adjustment phase 620, it is to be understood that the length of the initialization phase 602, maintenance phase 610 and adjustment phase 620 as depicted in FIG. 7 is not necessarily drawn to scale. It is further noted that the adjustment phase may be a specified number of ventricular beats which may include Vsense signals (counted as non-capture beats) and delivered CSP pulses (which may be determined as capture beats and/or non-capture beats).

At the termination of the adjustment phase 620, control circuit 80 determines a rate adjustment 618. The rate adjustment 618 may be applied to the adjustment phase CSP rate 616 and can be based on the number of CSP pulses that resulted in ventricular capture during the adjustment phase 620 and the CSP escape interval applied during the adjustment phase 620 (corresponding to the adjustment phase rate 616) or the ending CSP escape interval applied during the maintenance phase 610. The rate adjustment 618 may be an increase (as shown in this example) or a decrease or no change as described above and shown in the examples of TABLE I.

Control circuit 80 starts the next maintenance phase 630 at the updated maintenance phase CSP rate 632. The updated maintenance phase CSP rate 632 is determined from the adjustment phase CSP rate 616, adjusted by the rate adjustment 618, using the CSP escape interval offset 622. The updated maintenance phase CSP rate 632 may be determined by subtracting the offset 622, e.g., 50 ms, from the CSP escape interval corresponding to the adjustment phase CSP rate 616 adjusted by the rate adjustment 618. In other examples, the new maintenance phase CSP rate 632 may be determined by control circuit 80 by increasing the adjustment phase CSP rate 616 by subtracting the offset 622 (to obtain the previous ending maintenance phase CSP escape interval corresponding to rate 612) and subsequently adjusting the previous maintenance phase rate 612 by the rate adjustment 618. This process of controlling asynchronous CSP during subsequent maintenance phases and adjustment phases may continue as long as the atrial tachyarrhythmia is being detected.

FIG. 8 is diagram of an example CSP therapy programming window 700 that may be displayed as a GUI by external device 50 for enabling a user to select a CSP therapy command for transmission to pacemaker 14/114. In this example, the CSP therapy is delivered in the area of the LBB and is therefore referred to as “LBBAP therapy.” As such, in this illustrative example, a user may select the “LBBAP therapy” field 702 for selecting a LBBAP therapy command for transmission to pacemaker 14. For the sake of example, the CSP therapy options presented by the various windows and fields of a GUI represented in FIGS. 8-10 relate to pacing the His-Purkinje conduction system in the area of the LBB such that all CSP therapy labels in the diagrams of the example GUIs shown in the accompanying drawings are shown as “LBBAP therapy” labels. It is to be understood, however, that the CSP therapy may be delivered to other locations along the His-Purkinje conduction system and may be labeled as such in the various GUI windows and fields, e.g., His Bundle pacing therapy, RBBAP therapy, or more generally “CSP therapy” labels without specifying a location of the CSP site. For example, when the programming windows described herein are displayed as GUIs for programming leadless pacemaker 114 instead of pacemaker 14, “CSP therapy” labels may be used instead of “LBBAP therapy” labels because the pacing site may correspond to an area of the His Bundle instead of the LBB.

Upon user selection of the LBBAP therapy field 702 shown in FIG. 8, a menu of CSP therapy options may be displayed to enable the user to select the LBBAP therapy. The LBBAP therapy menu displayed in window 702, e.g., as a drop down list or in a pop-up window, may list at least three possible LBBAP therapies, the minimize LBBAP therapy, maximize LBBAP therapy, and complete AV block therapy. In the example shown in FIG. 8, the maximize LBBAP therapy is selected.

The selected maximize LBBAP therapy is displayed in the therapy field 702. The primary or permanent pacing mode may auto-populate in the pacing mode field 704 of the programming window 700 in response to the LBBAP therapy selection. In this case, for the maximize LBBAP therapy, control circuit 80 will control therapy delivery circuit 84 to deliver atrial synchronous LBBAP in a dual chamber pacing mode, which can be displayed as a DDD(R) pacing mode in field 704 (with the “R” designating that rate response pacing is enabled as shown in field 705). It is noted that a user may be able to turn on or off rate response pacing (e.g., in field 705) such that the permanent pacing mode may be displayed as DDDR or DDD depending on whether rate response pacing is turned on or not.

When the user selects the maximize LBBAP therapy in field 702, the display unit 54 may display various fields pertaining to the maximize LBBAP therapy. For instance, an AV pacing interval window 752 may display a field 755 for programming the percentage of the SAV and PAV conduction times that is used by control circuit 80 for setting and updating the SAV and PAV pacing intervals during the DDDR pacing mode. The percentage may or may not be programmable by a user. In some examples, the default percentage displayed in field 755 is 56%, which may be an optimal percentage of the SAV and PAV conduction times for achieving improved ventricular function during the LBBAP therapy. For example, 56% of the measured intrinsic AV conduction time can be associated with minimized ventricular activation times (e.g., narrowest QRS width) indicating synchrony between the left and right ventricles and maximized left ventricular dP/dt (rate of pressure generation during ventricular systole). Decreased or minimized ventricular activation time and increased or maximized left ventricular dP/dt measurements are strong indicators of optimized ventricular function. Decreased ventricular activation time and increased left ventricular dP/dt measurements can be used for selecting an optimal percentage of the intrinsic AV conduction times for setting the SAV and PAV pacing intervals by control circuit 80 for promoting the greatest clinical benefit of the LBBAP (or more generally CSP) therapy to the patient. The specified percentage shown in field 755 may be a fixed value identified to be optimal in a population of patients, e.g., based on minimized ventricular activation time and maximized left ventricular dP/dt. In other examples, the specified percentage shown in field 755 may be user programmable, e.g., over a range of 40% to 60%, for tailoring optimal SAV and PAV pacing interval for a given patient. The percentage displayed in field 755 is unique to the programming window 700 generated as a GUI by external device 50 when maximize CSP therapy, in this example maximize LBBAP therapy, is selected because a percentage of the measured AV conduction time is not used by control circuit 80 when the minimize CSP therapy or the complete AV block therapy is selected.

As shown in AV pacing interval programming window 752 of FIG. 8, a user may select maximum AV pacing interval(s) in fields 754. Fields 754 may enable a user to select or enter separately programmable values for the maximum limits of the SAV and PAV pacing intervals. The PAV and SAV pacing interval maximum limits may be set to 150 ms and 120 ms, respectively, as non-limiting examples. A range of available values, e.g., between 100 and 200 ms, may be available for programming the maximum limits of the SAV and PAV pacing intervals in fields 754.

When control circuit 80 of pacemaker 14 receives the maximize LBBAP therapy command from external device 50, control circuit 80 will enable AV conduction checks and update the SAV and PAV pacing intervals to the specified percentage (displayed in field 755) of the measured SAV conduction times and PAV conduction times, respectively. The SAV and PAV pacing intervals automatically updated by control circuit 80 to the specified a percentage (displayed in field 755) of measured SAV and PAV conduction times will not exceed the programmed maximum SAV and maximum PAV pacing intervals, respectively, as entered and/or displayed in fields 754.

The user may be able to turn rate adaptive AV pacing intervals on or off in field 756. In this example, rate adaptive AV pacing interval is shown as being turned on. When turned on, control circuit 80 may automatically adjust the SAV and PAV pacing intervals with changes in heart rate, e.g., with changes in sensed and/or paced PPIs. Control parameters that may be programmed into pacemaker 14 (or pacemaker 114) for controlling heart rate based adjustments to the SAV and PAV pacing intervals may include a start rate, stop rate, minimum SAV pacing interval and/or minimum PAV pacing interval. Control circuit 80 may perform adjustment of the PAV and SAV pacing intervals over a range of user programmed or default heart rates. The range of heart rates over which control circuit 80 may adjust the PAV and SAV pacing intervals are defined by a start rate and a stop rate as displayed in fields 758a and 758b, respectively. The start and stop rates may be 90 bpm and 130 bpm, respectively, as illustrative, non-limiting examples. A start rate and a stop rate may be programmed to other heart rates, or a different default start and/or stop rate than the values shown may be displayed. For example, the start heart rate may be between 70 to 110 bpm as non-limiting examples, and the stop rate may be between 110 and 150 bpm as non-limiting examples.

Control circuit 80 may be configured to adjust the SAV and PAV pacing intervals linearly, for example, from their respective starting values (e.g., the specified percentage of the most recently measured SAV and PAV conduction times) as heart rate increases above the start rate. When the heart rate is less than the start rate, the PAV and SAV pacing intervals are the specified percentage of the most recently measured PAV and SAV conduction times (up to the maximum SAV and PAV pacing intervals 754). As heart rate increases above the start rate (field 758a), the SAV and PAV pacing intervals may be decreased by control circuit 80 from their starting values to no less than the minimum values displayed in field 760. As heart rate decreases back toward the start rate, the SAV and PAV pacing intervals may be increased back toward their starting values. When the heart rate is equal to or greater than the stop rate, the PAV and SAV pacing intervals are not less than the minimum PAV and minimum SAV displayed in fields 760. As such, when a user turns on rate adaptive AV pacing intervals in field 756, control circuit 80 receives the programming command to enable rate-based adjustments of the PAV and SAV pacing intervals.

In some examples, the programming window 700 may include a field 714 to enable a user to turn on (enable) or turn off (disable) automatic rate adjustments during asynchronous CSP during a detected fast atrial rate or atrial tachyarrhythmia, e.g., during AF. When the automatic rate adjustment is enabled, control circuit 80 may operate according to the methods described above, e.g., in conjunction with FIGS. 6 and 7, to determine maintenance phase CSP rates to promote CSP at a rate that encourages capture of the ventricles at a regular rate. The user may be able to select a maximum CSP rate in field 714. The maximum rate may be between 90 and 120 bpm with a default value of 110 bpm as an example, but other maximum rates may be selectable and programmable. The maintenance phase CSP rate may be adjusted up to but not greater than the programmed maximum rate in field 714. Automatic pacing rate adjustments by control circuit 80 for increasing the likelihood of capture by the LBBAP pulses (or more generally CSP pulses) during atrial tachyarrhythmia to promote ventricular rate regularity may be available for turning on or off (e.g., default setting may be on) by a user for the maximize CSP therapy and may be available for all selectable CSP therapies.

The programming window 700 may display other user programmable parameters that can be transmitted to pacemaker 14 (or leadless pacemaker 114) for use in controlling the selected CSP therapy, in this instance the maximize LBBAP therapy. For example, other pacing control parameters that may be programmable in programming window 700 but are not shown in FIG. 8 include a lower pacing rate, an upper tracking rate, and an upper sensor rate (for limiting the maximum rate response pacing rate).

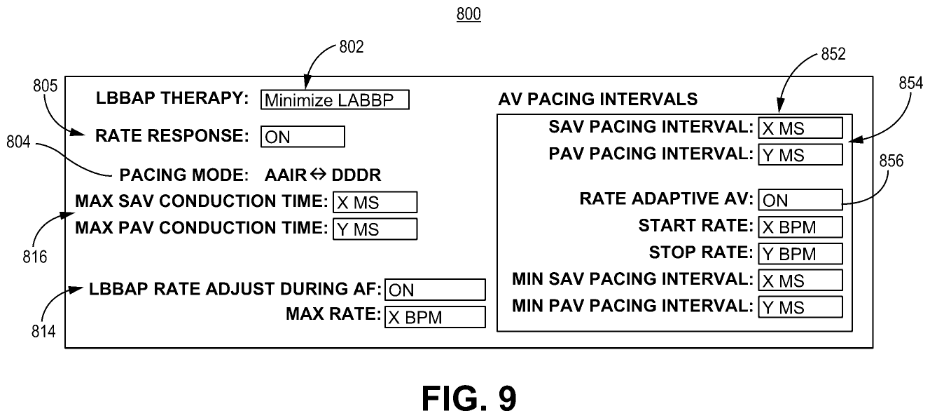

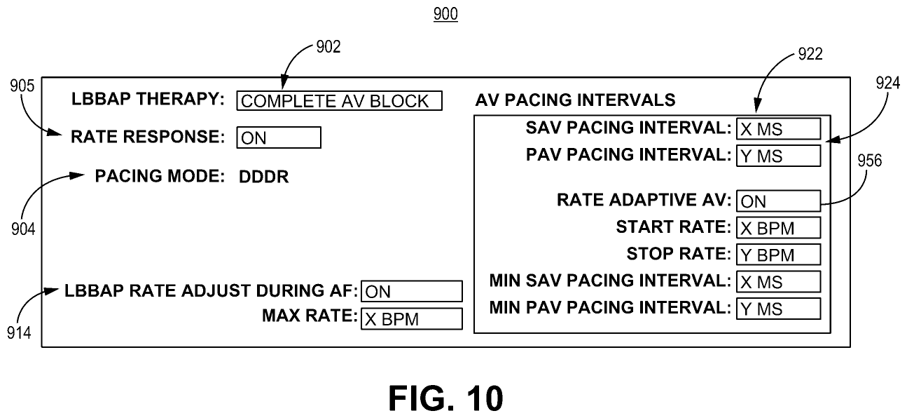

While not explicitly shown in FIG. 8, it is to be understood that the programming window 700 (and other programming windows shown and described herein, e.g., in FIGS. 9 and 10) may include various windows, fields, menus, etc. to enable a user to program other atrial pacing and sensing control parameters and CSP and ventricular sensing control parameters. Other user programmable control parameters may include the atrial and CSP pulse amplitudes, pacing pulse widths, atrial sensitivity for sensing intrinsic P-waves, and ventricular sensitivity for sensing intrinsic R-waves, as examples.

Programming windows displayed by external device 50 may enable a user to program an atrial tachycardia and/or atrial fibrillation (AT/AF) detection interval and ventricular tachycardia and/or ventricular fibrillation (VT/VF) detection interval used by control circuit 80 for monitoring for and detecting arrhythmias. As described herein, detection of an atrial tachyarrhythmia, e.g., AF, may cause control circuit 80 to switch to an asynchronous CSP mode with automatic rate adjustments (if enabled) to promote effective CSP pulses that capture the ventricles and control the ventricular rate during AF, which may otherwise conduct to the ventricles causing rapid and/or irregular RRIs. A user may be able to program therapies that can be delivered in response to detecting an arrhythmia in some examples, e.g., when pacemaker 14 includes CV/DF shock capabilities and/or anti-tachycardia pacing capabilities.

FIG. 9 is a diagram of a programming window 800 that may be displayed in a GUI when the LBBAP therapy is selected to be the minimize LBBAP therapy as shown in field 802. As described above, a user may select minimize LBBAP therapy from a menu of available CSP therapies when the LBBAP therapy field 802 is selected in the programming window 800. When the minimize LBBAP therapy is selected, the external device 50 may display the pacing mode in field 804 as being a single chamber atrial pacing mode, e.g., AAIR or AAI (depending on whether rate response pacing is turned on in field 805). The AAI(R) pacing mode may be referred to as the primary or permanent pacing mode during minimize LBBAP. However, external device 50 may further display an arrow and a dual chamber pacing mode, e.g., DDD(R) pacing mode, in field 804 to indicate that the pacing mode may be switched to a dual chamber pacing mode for delivering atrial synchronous CSP, in this case atrial synchronous LBBAP, when the intrinsic AV conduction time exceeds a maximum limit. As such the pacing mode may be switched from the single chamber atrial pacing mode to include LBBAP when a measured SAV or PAV conduction time exceeds a maximum limit, which may be programmable as shown in fields 816.

When the PAV conduction time exceeds the programmed maximum limit, LBBAP will be enabled by control circuit 80, e.g., by switching from the single chamber atrial pacing mode, e.g., AAI(R) pacing mode, to the dual chamber CSP mode, e.g., DDD(R) pacing mode. The PAV conduction time maximum limit may be programmable to between 230 and 300 ms as non-limiting examples. If the SAV conduction time exceeds the programmed maximum limit, LBBAP will be enabled by control circuit 80, e.g., by switching from the AAI(R) pacing mode to the DDD(R) pacing mode. The SAV conduction time maximum limit may be programmable to between 230 and 300 ms as non-limiting examples. As generally described above, e.g., in conjunction with FIG. 4, control circuit 80 enables AV conduction checks and determines when a measured AV conduction time is greater than the programmed maximum AV conduction time limits for switching between the single chamber atrial pacing mode (to promote intrinsic AV conduction and minimize CSP) to an atrial synchronous CSP mode when AV conduction is slow or AV block is present and atrial synchronous CSP can promote improved AV synchrony.

In response to receiving the programmed maximum limits of the PAV and SAV conduction times, control circuit 80 may determine a PAV pacing interval and a SAV pacing interval as a specified percentage, e.g., 56%, of the maximum limits of the respective PAV and SAV conduction times (fields 816). External device 50 may be configured to auto-populate the PAV pacing interval and SAV pacing interval displayed in field 854 as the specified percentage of the PAV and SAV conduction time maximum limits (field 816) that will be determined and used by control circuit 80 during the atrial synchronous CSP mode (e.g., DDD(R) mode). In some examples, the PAV and SAV pacing intervals displayed in field 854 are not user programmable. They are set by control circuit 80 based on the programmed maximum limits entered in fields 816 and received by control circuit 80 when the programming commands are transmitted from external device 50 to pacemaker 14. The percentage of the maximum AV conduction time limits used by control circuit 80 to set the PAV and SAV pacing intervals may be a fixed percentage, e.g., 56%, of the intrinsic AV conduction time found to maximize left ventricular dP/dt and minimize QRS width as a metric of ventricular synchrony. In other examples, a user may override the auto-populated maximum PAV and SAV pacing intervals displayed in field 854 and program patient specific values for the PAV and SAV pacing intervals used by control circuit 80 when the measured PAV and SAV conduction times during AV conduction checks exceed the displayed maximum PAV and SAV conduction time limits (fields 816).

Window 800 may further display options for a user to enable rate adaptive AV pacing intervals in field 856 of the AV pacing intervals field 852. As generally described above in conjunction with FIG. 8, control circuit 80 may decrease the SAV pacing interval or the PAV pacing interval as the heart rate increases above the start rate. The SAV pacing interval is not shortened to less than a minimum SAV pacing interval, and the PAV pacing interval is not shortened to less than the minimum PAV pacing interval when the heart rate is equal to the stop rate or higher. During the minimize CSP therapy, the starting PAV and SAV pacing intervals are the values shown in fields 854, which may be user-programmed values or a specified percentage of the user programmed maximum AV conduction time limits (fields 816). The start rate, stop rate, minimum SAV pacing interval and minimum PAV pacing interval may all be programmable by a user in the AV pacing interval window 852 as shown in FIG. 9.

FIG. 10 is a diagram of a programming window 900 when the complete AV block therapy is selected and displayed in LBBAP therapy field 902. When the complete AV block therapy is selected, the pacing mode field 904 may auto-populate to display that the primary or permanent pacing mode is a dual chamber pacing mode providing atrial synchronous LBBAP, e.g., in a DDDR pacing mode when rate response pacing is enabled (field 905) or in a DDD pacing mode when rate response pacing is not enabled.

When the programmed therapy is complete AV block therapy, control circuit 80 disables AV conduction checks as described above. As such, the PAV and SAV pacing intervals may be selected in fields 924. The PAV and SAV pacing intervals are not updated based on a percentage of measured AV conduction times (due to complete AV block precluding changes in AV conduction time) but may be adjusted when rate adaptive AV pacing interval adjustments is turned on (field 956) as generally described above. In this case however, fields 816 of FIG. 9 are not displayed when complete AV block therapy is selected because AV conduction checks are disabled. Rather, the programmed or default PAV and SAV pacing intervals can be displayed in fields 924 and the start rate, stop rate, and minimum PAV and SAV pacing intervals may be displayed in respective fields in the AV pacing interval programming window 922, as generally described above, for use by control circuit 80 in controlling the AV pacing intervals during the complete AV block therapy. Control circuit 80 may automatically adjust the AV pacing intervals from the programmed starting values in fields 924 to as short as the minimum AV pacing intervals shown in field 922 based on heart rate. However, adjustments of the AV pacing intervals based on intrinsic AV conduction time measurements during the complete AV block therapy is disabled by control circuit 80.

Automatic LBBAP rate adjustments may be turned on by the user in field 914 to enable control circuit 80 to adjust the CSP escape intervals during a detected AF episode as generally described above in conjunction with FIGS. 5-7. Switching between a single chamber and dual chamber pacing mode (as enabled during the minimize LBBAP therapy), which is also based on measured intrinsic AV conduction times, is disabled during the complete AV block mode. Other fields relating to pacing control parameters and sensing control parameters which may be displayed in window 900, but are not shown for the sake of clarity, are generally described above, such as the atrial pacing and LBBAP pulse amplitudes, pulse widths, pace polarity, P-waves sensitivity, R-wave sensitivity and so on.

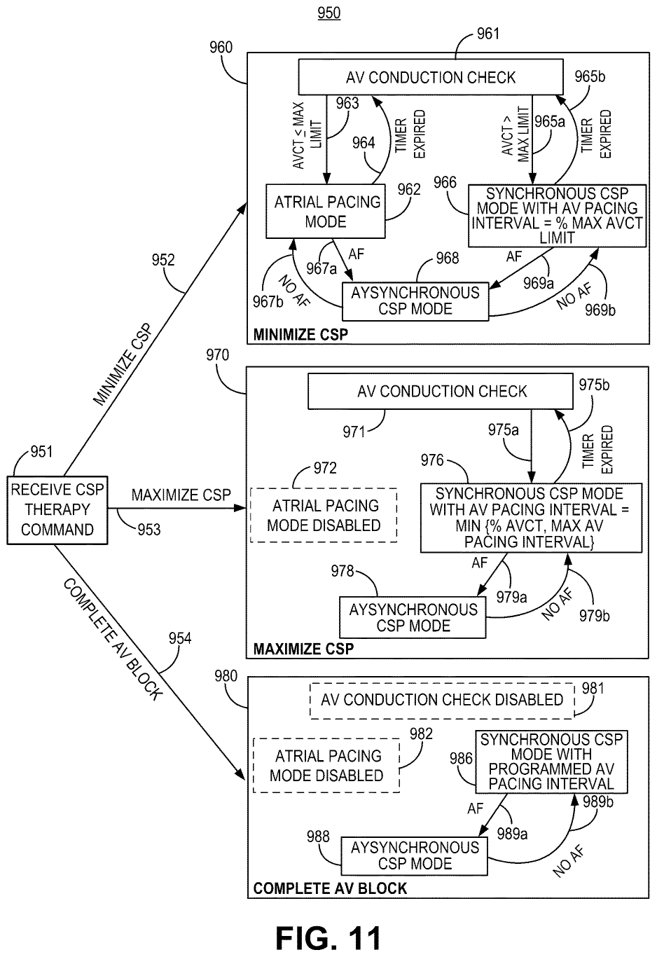

FIG. 11 is a diagram 950 of pacing mode operating states of control circuit 80 that control pacemaker 14/114 functions based on a received CSP therapy command according to some examples. Control circuit 80 receives a CSP therapy command at block 951. As described above, the CSP therapy command may be received by pacemaker telemetry circuit 88 (FIG. 2) from an external device, e.g., external device 50 shown in FIG. 1A. The CSP therapy command may be received as one of a minimize CSP therapy command, a maximum CSP therapy command or a complete AV block therapy command.

When the received CSP therapy command is minimize CSP therapy (arrow 952) control circuit 80 enables the pacing mode states and control parameters for the minimize CSP therapy as generally represented in block 960. Control circuit 80 enables AV conduction checks, a single chamber atrial pacing mode and an atrial synchronous CSP mode for controlling operations during the minimize CSP therapy. Control circuit 80 may control therapy delivery circuit 84 according to four different pacing operating states including the AV conduction check state (block 961), the atrial pacing mode state (block 962), the dual chamber pacing mode state (block 966) and the non-tracking, asynchronous CSP mode state (block 968).

Control circuit 80 may perform an AV conduction check in state 961 to determine the intrinsic AV conduction time, which may include determining an SAV conduction time and a PAV conduction time as described above. When the intrinsic AV conduction time is less than or equal to a programmed AV conduction time (AVCT) maximum limit, which may be a respective maximum SAV conduction time or maximum PAV conduction time, control circuit 80 transitions (arrow 963) to the atrial pacing mode state of block 962. Control circuit 80 controls therapy delivery circuit 84 to deliver single chamber atrial pacing, e.g., AAI (or AAIR if rate response pacing is enabled). When the AV conduction check timer expires (as described above), control circuit 80 transitions (arrow 964) from the atrial pacing mode state of block 962 to the AV conduction check state of block 961. If the intrinsic AVCT is still less than or equal to the maximum AVCT threshold, control circuit 80 can transition back to the atrial pacing mode state of block 962.

If the intrinsic AVCT is greater than the maximum AVCT limit, control circuit 80 may transition (arrow 965a) from the AV conduction check state of block 961 to the synchronous CSP mode state of block 966. Control circuit 80 may switch the pacing mode to a dual chamber atrial synchronous CSP mode, e.g., DDD (or DDDR when rate response pacing is enabled). Therapy delivery circuit 84 may deliver atrial pacing as needed and deliver CSP pulses at an AV pacing interval from Asense event signals or atrial pacing pulses as controlled by control circuit 80. As described above, the AV pacing intervals may be applied as an SAV pacing interval or a PAV pacing interval. Control circuit 80 may determine the AV pacing interval as a specified percentage of the maximum AV conduction time limit (% MAX AVCT LIMIT as indicated in block 966). For instance, control circuit 80 may apply the SAV pacing interval or PAV pacing interval for controlling the timing of atrial synchronous CSP pulses as a specified percentage of the maximum SAV conduction time limit or the maximum PAV conduction time limit, respectively. In other examples, the AV pacing intervals may be applied according to user programmed values.

When the AV conduction check timer expires, control circuit 80 may transition back to the AV conduction check state of block 961 (see transition arrow 965b). Control circuit 80 may redetermine the intrinsic AV conduction time for controlling when the pacing mode is switched back to the single chamber atrial pacing mode state (block 962), e.g., when the intrinsic AV conduction time is less than a maximum AV conduction time limit.

At any time during the single chamber atrial pacing mode state of bock 962 or the synchronous CSP mode state of block 966, control circuit 80 may detect an atrial tachyarrhythmia, e.g., AF, using any implemented atrial tachyarrhythmia detection method as discussed above. In response to detecting atrial tachyarrhythmia, control circuit 80 may switch from the single chamber atrial pacing mode or the atrial synchronous CSP mode, whichever is currently in effect (block 962 or 966), to a non-tracking asynchronous CSP mode state of block 968 (see transition arrows 967a and 969a). For example, control circuit 80 may switch the pacing mode from AAI(R) (block 962) to a DDI(R) pacing mode or from DDD(R) (block 966) to a DDI(R) pacing mode. During the asynchronous CSP mode state of block 968, atrial pacing may remain available in a dual chamber asynchronous CSP mode but is likely to be inhibited by control circuit 80 due to the fast atrial rate. CSP may be delivered according to a programmed ventricular lower rate with rate adjustments as needed if rate adjustments during atrial tachyarrhythmia is enabled (according to the methods described above in conjunction with FIGS. 5-7). When atrial tachyarrhythmia is no longer being detected, control circuit 80 may transition back to whichever pacing mode state (block 926 or block 966) that was in effect at the time of detecting the atrial tachyarrhythmia (see transition arrows 967b and 969b).

During the atrial synchronous CSP mode state of block 966, control circuit 80 may limit the CSP rate to an upper tracking rate limit. When the sensed atrial rate is faster than an upper tracking rate limit, control circuit 80 may withhold or delay a CSP pulse to avoid delivering the CSP pulse at rate from the preceding most recent CSP pulse that is faster than the upper tracking rate limit.

When the CSP therapy command received at block 951 is the maximize CSP therapy (as indicated by arrow 953), control circuit 80 may enable the pacing modes and pacing control parameters for the maximize CSP therapy as generally represented in block 970. Control circuit 80 enables AV conduction checks and a synchronous CSP mode as the primary or permanent pacing mode for controlling operations during the maximize CSP therapy. The single chamber atrial pacing mode state, available when the minimize CSP therapy is selected, is disabled by control circuit 80 as indicated by dashed box 972. During the maximize CSP therapy, control circuit 80 may control therapy delivery circuit 84 according to three different pacing states including the AV conduction check state (block 971), the synchronous CSP mode state (block 976) and the asynchronous CSP mode state (block 978).

Control circuit 80 may perform an AV conduction check in state 971 to determine the intrinsic AV conduction time, which may include determining an SAV conduction time and a PAV conduction time as described above. Instead of using the AV conduction time to control switching between the single chamber atrial pacing mode state and the synchronous CSP mode state as described in conjunction with the minimize CSP therapy (block 960), control circuit 80 may use the measured intrinsic AV conduction times for setting the SAV pacing interval and the PAV pacing interval. Unlike the minimize CSP therapy, atrial synchronous CSP can be delivered when the AV conduction time is less than a maximum AV conduction time limit; the maximum AV conduction time limit that is applied to the measured AVCTs during the minimize CSP therapy may not be used or applied by control circuit 80 during the maximize CSP therapy.

Instead, control circuit 80 determines the SAV and PAV pacing intervals as a specified percentage (e.g., 56%) of the respective intrinsic SAV and PAV conduction times determined during the AV conduction check state (block 971). Control circuit 80 transitions to block 976 when the AV conduction check is complete (see arrow 975a). Therapy delivery circuit 84 may deliver atrial pacing as needed during the synchronous CSP mode (e.g., when an atrial pacing interval expires without an Asense signal from sensing circuit 86) and delivers CSP pulses at the determined SAV pacing interval from Asense signals or the determined PAV pacing interval from atrial pacing pulses. The SAV pacing interval and PAV pacing interval, therefore, can be updated by control circuit 80 based on the measured intrinsic SAV and PAV conduction times, respectively, when AV conduction checks are performed. For example, when the AV conduction check timer expires (see arrow 975b), control circuit 80 transitions back to the AV conduction check state of block 971. As shown in synchronous CSP state of block 976, the AV (SAV and/or PAV) pacing intervals may be set to a maximum AV pacing interval limit (e.g., as programmed in fields 754 of FIG. 8) or the specified percentage of the measured intrinsic AV conduction time, whichever is shorter.

At any time during the synchronous CSP mode state of block 976, control circuit 80 may detect an atrial tachyarrhythmia, e.g., AF, using any implemented atrial tachyarrhythmia detection method. In response to detecting atrial tachyarrhythmia, control circuit 80 may switch from the synchronous CSP mode state (block 976) to an asynchronous CSP mode state of block 978 (see arrow 979a). For example, control circuit 80 may switch from a DDD(R) pacing mode (block 966) to a DDI(R) pacing mode (block 978). During the asynchronous CSP mode state, atrial pacing may remain available in the dual chamber pacing mode but is likely to be inhibited due to the fast atrial rate. CSP may be delivered according to a programmed ventricular lower rate (which may be adjusted to a temporary LRI when rate response pacing is enabled). During the maximize LBBAP therapy, if automatic rate adjustment during atrial tachyarrhythmia is enabled, control circuit 80 may automatically adjust the CSP rate to promote capture of the ventricles and a regular ventricular rate, e.g., according to any of the methods described above in conjunction with FIGS. 5-7. When atrial tachyarrhythmia is no longer being detected, control circuit 80 may transition back to block 976 (arrow 979b).

During the synchronous CSP mode state of block 976, control circuit 80 may delay or inhibit a CSP pulse when the sensed atrial rate is faster than an upper tracking rate limit, as generally described above. A CSP pulse scheduled at the expiration of an SAV or PAV pacing interval that would result in a ventricular rate interval from the most recent preceding Vsense signal or CSP pulse may be withheld, inhibited or delayed so that a CSP is not delivered at a rate faster than the upper tracking rate limit.

When the CSP therapy command received at block 951 is the complete AV block therapy (as indicated by arrow 954), control circuit 80 may enable the pacing modes and pacing control parameters for the complete AV block therapy as generally represented in block 980. Control circuit 80 disables AV conduction checks as indicated by the dashed box 981. Control circuit 80 enables the synchronous CSP mode as the primary or permanent pacing mode state for controlling operations during the complete AV block therapy. The single chamber atrial pacing mode state available when the minimize CSP therapy is selected is disabled by control circuit 80 as indicated by dashed box 982. During the complete AV block therapy, control circuit 80 may control therapy delivery circuit 84 according to two different pacing states including the synchronous CSP mode state (block 986) and the asynchronous CSP mode state (block 988).

No AV conduction checks are performed during the complete AV block therapy. Control circuit 80 applies programmed SAV and PAV pacing intervals for controlling the timing of the atrial synchronous CSP pulses while operating in the synchronous CSP mode state (block 986). The pacing mode may be a rate response pacing mode (e.g., DDDR). The SAV and PAV pacing intervals may be adjustable with changes in heart rate as described above, e.g., in conjunction with FIG. 8, between a start heart rate and stop heart rate. Therapy delivery circuit 84 may deliver atrial pacing as needed during the dual chamber pacing mode (e.g., when an atrial pacing interval expires without an Asense signal from sensing circuit 86) and delivers CSP pulses at the programmed SAV pacing interval from Asense event signals or the programmed PAV pacing interval from atrial pacing pulses.

At any time during the synchronous CSP mode state of block 986, control circuit 80 may detect an atrial tachyarrhythmia, e.g., AF, and transition (see arrow 989a) to the asynchronous CSP mode state of block 988. Control circuit 80 may perform automatic CSP rate adjustments during a detected tachyarrhythmia, e.g., as described in conjunction with FIGS. 5-7 above. Control circuit 80 may transition back to the synchronous CSP mode state of block 986 when the atrial tachyarrhythmia is no longer being detected (see arrow 989b).

Further disclosed herein is the subject matter of the following examples:

Example 1. A medical device including a sensing circuit configured to sense at least one cardiac electrical signal and a therapy delivery circuit configured to generate atrial pacing pulses and conduction system pacing pulses. The medical device may further include a communication circuit configured to receive a conduction system pacing therapy command and a control circuit configured to receive the conduction system pacing therapy command from the communication circuit, wherein the conduction system pacing therapy command corresponds to one of a first conduction system pacing therapy command or a second conduction system pacing therapy command. The control circuit may be further configured to determine an AV pacing interval according to a first method when the conduction system pacing therapy command is the first conduction system pacing therapy command and determine the AV pacing interval according to a second method different than the first method when the conduction system pacing therapy command is the second conduction system pacing therapy command. The control circuit may be further configured to schedule conduction system pacing pulses for delivery by the therapy delivery circuit at the determined AV pacing interval and according to the received one of the first conduction system pacing therapy command or the second conduction system pacing therapy command.

Example 2. The medical device of example 1 wherein the control circuit is further configured to determine an AV conduction time from the at least one cardiac electrical signal and determine the AV pacing interval according to the second method different by determining the AV pacing interval as a first percentage of the determined AV conduction time.

Example 3. The medical device of one of examples 1 or 2 wherein the control circuit is further configured to determine an AV conduction time from the at least one cardiac electrical signal, compare the AV conduction time to a maximum AV conduction time, and determine the AV pacing interval according to the first method by determining the AV pacing interval as a second percentage of the maximum AV conduction time limit. The control circuit may be further configured to, when the received conduction system pacing therapy command is the first conduction system pacing therapy command and the AV conduction time is greater than the maximum AV conduction time, schedule the conduction system pacing pulses for delivery by the therapy delivery circuit at the AV pacing interval determined as the percentage of a maximum AV conduction time limit.

Example 4. The medical device of example 3 wherein the control circuit is further configured to determine the AV pacing interval according to the first method by determining the AV pacing interval as the second percentage of the maximum AV conduction time limit where the second percentage equals the first percentage.

Example 5. The medical device of any one of examples 1-2 wherein the communication circuit is further configured to receive a user programmed AV pacing interval, and the control circuit is further configured to determine the AV pacing interval according to the first method by determining the AV pacing interval from the received user programmed AV pacing interval.

Example 6. The medical device of any one of examples 2-5 wherein the control circuit is further configured to, when the conduction system pacing therapy command is the first conduction system pacing therapy command, withhold scheduling the conduction system pacing pulses by switching to a single chamber atrial pacing mode when the AV conduction time is less than the maximum AV conduction time limit.

Example 7. The medical device of any one of examples 1-6 wherein the control circuit is further configured to, in response to the received conduction system pacing therapy command being the first conduction system pacing therapy command, enable AV conduction checks for detecting AV block and control the therapy delivery circuit to deliver atrial pacing pulses in a single chamber atrial pacing mode. The control circuit may switch from the single chamber atrial pacing mode to an atrial synchronous conduction system pacing mode in response to AV conduction block being detected from an AV conduction check and schedule the conduction system pacing pulses for delivery by the therapy delivery circuit at the AV pacing interval after switching to the atrial synchronous conduction system pacing mode.

Example 8. The medical device of any one of examples 1-7 wherein the control circuit is further configured to, in response to the received conduction system pacing therapy command being the second conduction system pacing therapy command, enable AV conduction checks for measuring AV conduction times and disable a single chamber atrial pacing mode. The control circuit may control the therapy delivery circuit to deliver the conduction system pacing pulses at the AV pacing interval during an atrial synchronous conduction system pacing mode.

Example 9. The medical device of any one of examples 1-8 wherein the control circuit is further configured to, when the conduction system pacing therapy command is the second conduction system pacing therapy command, determine that the percentage of the AV conduction time is greater than a maximum AV pacing interval limit and adjust the AV pacing interval to the maximum AV pacing interval limit when the percentage of the AV conduction time is greater than the maximum AV pacing interval limit.

Example 10. The medical device of any one of examples 1-9 wherein the control circuit is further configured to receive a conduction system pacing therapy command corresponding to a third conduction system pacing therapy command and, in response to receiving the third conduction system pacing therapy command, disable AV conduction checks, disable a single chamber atrial pacing mode and schedule conduction system pacing pulses for delivery by the therapy delivery circuit at a programmed AV pacing interval.

Example 11. The medical device of any one of examples 1-10 wherein the control circuit is further configured to determine an atrial rate from at least one of atrial pacing pulses delivered by the therapy delivery circuit or the at least one cardiac electrical signal and adjust the AV pacing interval based on the atrial rate.

Example 12. The medical device of any one of examples 1-11 wherein the control circuit is further configured to detect atrial tachyarrhythmia from the at least one cardiac electrical signal and, in response to detecting the atrial tachyarrhythmia, control the therapy delivery circuit to deliver the conduction system pacing pulses at a conduction system pacing rate according to an atrial asynchronous conduction system pacing mode.

Example 13. The medical device of example 12 wherein the control circuit is further configured to determine that at least one of the conduction system pacing pulses delivered at the conduction system pacing rate does not capture cardiac tissue and adjust the conduction system pacing rate in response to determining that the at least one conduction system pacing pulse does not capture cardiac tissue.

Example 14. The medical device of one of examples 12 or 13 wherein the control circuit is further configured to adjust the conduction system pacing rate during an initialization phase where an initialization phase ending pacing escape interval is reached at which the conduction system pacing pulses are capturing the cardiac tissue, set a first maintenance phase pacing escape interval to be a specified time interval shorter than the initialization phase ending pacing escape interval and, for a maintenance time interval, schedule delivery of the conduction system pacing pulses according to the first maintenance phase pacing escape interval.

Example 15. The medical device of example 14 wherein the control circuit is further configured to upon expiration of the maintenance phase, increase the maintenance phase pacing escape interval by the specified time interval to an adjustment phase pacing escape interval, schedule conduction system pacing pulses at the adjustment phase pacing escape interval, determine a count of the conduction system pacing pulses that are delivered by the therapy delivery circuit at the adjustment phase pacing escape interval that capture cardiac tissue, and adjust the conduction system pacing rate based on the count. The control circuit may set a second maintenance phase pacing escape interval to a rate interval corresponding to the adjusted conduction system pacing rate less the specified time interval.

Example 16. The medical device of any one of examples 14 or 15 wherein the sensing circuit is further configured to sense ventricular event signals attendant to intrinsic ventricular depolarizations from the at least one cardiac electrical signal. The control circuit may be further configured to decrease the conduction system pacing rate by decreasing the first maintenance phase pacing interval in response to the sensing circuit sensing at least one ventricular event signal during the maintenance phase.

Example 17. A method including sensing at least one cardiac electrical signal,

receiving one of a first conduction system pacing therapy command or a second conduction system pacing therapy command, determining an AV pacing interval according to a first method in response to receiving the first conduction system pacing therapy command and determining the AV pacing interval according to a second method different than the first method in response to receiving the second conduction system pacing therapy command. The method may further include scheduling conduction system pacing pulses for delivery at the determined AV pacing interval and according to the received one of the first conduction system pacing therapy command or the second conduction system pacing therapy command.

Example 18. The method of example 17 further including determining an AV conduction time from the at least one cardiac electrical signal and determining the AV pacing interval according to the second method by determining the AV pacing interval as a first percentage of the determined AV conduction time.

Example 19. The method of one of examples 17 or 18 further including determining an atrioventricular (AV) conduction time from the at least one cardiac electrical signal, comparing the AV conduction time to a maximum AV conduction time and determining the AV pacing interval according to the first method by determining the AV pacing interval as a second percentage of the maximum AV conduction time limit. The method may further include, in response to receiving the first conduction system pacing therapy command and the AV conduction time being greater than the maximum AV conduction time, scheduling the conduction system pacing pulses at the AV pacing interval determined as the percentage of a maximum AV conduction time limit.

Example 20. The method of example 19 further including determining the AV pacing interval according to the first method by determining the AV pacing interval as the second percentage of the maximum AV conduction time limit where the second percentage equals the first percentage.

Example 21. The method of any one of examples 17-18 further including receiving a user programmed AV pacing interval and determining the AV pacing interval according to the first method by determining the AV pacing interval from the received user programmed AV pacing interval.

Example 22. The method of any one of examples 17-21 further including, in response to receiving the first conduction system pacing therapy command, withholding scheduling the conduction system pacing pulses by switching to a single chamber atrial pacing mode when the AV conduction time is less than the maximum AV conduction time limit.

Example 23. The method of any one of examples 17-22 further including, in response to receiving the first conduction system pacing therapy command, enabling AV conduction checks for detecting AV block, delivering atrial pacing pulses in a single chamber atrial pacing mode and switching from the single chamber atrial pacing mode to an atrial synchronous conduction system pacing mode in response to AV conduction block being detected from an AV conduction check. The method may further include scheduling the conduction system pacing pulses at the AV pacing interval after switching to the atrial synchronous conduction system pacing mode.

Example 24. The method of any one of examples 17-23 further including, in response to receiving the second conduction system pacing therapy, enabling AV conduction checks for measuring AV conduction times, disabling a single chamber atrial pacing mode and delivering the conduction system pacing pulses at the AV pacing interval during an atrial synchronous conduction system pacing mode.

Example 25. The method of any one of examples 17-24 further including, in response to receiving the second conduction system pacing therapy command determining that the percentage of the AV conduction time is greater than a maximum AV pacing interval limit and adjusting the AV pacing interval to the maximum AV pacing interval limit when the percentage of the AV conduction time is greater than the maximum AV pacing interval limit.

Example 26. The method of any one of examples 17-25 further including receiving a third conduction system pacing therapy command and, in response to receiving the third conduction system pacing therapy command, disabling AV conduction checks, disabling a single chamber atrial pacing mode and scheduling conduction system pacing pulses at a programmed AV pacing interval.

Example 27. The method of any one of examples 17-26 further including determining an atrial rate from at least one of delivered atrial pacing pulses or the at least one sensed cardiac electrical signal and adjusting the AV pacing interval based on the atrial rate.

Example 28. The method of any one of examples 17-27 further including detecting atrial tachyarrhythmia from the at least one cardiac electrical signal and, in response to detecting the atrial tachyarrhythmia, delivering the conduction system pacing pulses at a conduction system pacing rate according to an atrial asynchronous conduction system pacing mode.

Example 29. The method of example 28 further including determining that at least one of the conduction system pacing pulses delivered at the conduction system pacing rate does not capture cardiac tissue and adjusting the conduction system pacing rate in response to determining that the at least one conduction system pacing pulse does not capture cardiac tissue.

Example 30. The method of one of examples 28 or 29 further including adjusting the conduction system pacing rate during an initialization phase where an initialization phase ending pacing escape interval is reached at which the conduction system pacing pulses are capturing the cardiac tissue and setting a first maintenance phase pacing escape interval to be a specified time interval shorter than the initialization phase ending pacing escape interval. The method may further include, for a maintenance time interval, scheduling delivery of the conduction system pacing pulses according to the first maintenance phase pacing escape interval.

Example 31. The method of example 30 further including upon expiration of the maintenance phase, increasing the maintenance phase pacing escape interval by the specified time interval to an adjustment phase pacing escape interval and scheduling conduction system pacing pulses at the adjustment phase pacing escape interval. The method may further include determining a count of conduction system pacing pulses that are delivered at the adjustment phase pacing escape interval that capture cardiac tissue and adjusting the conduction system pacing rate based on the count. The method may further include setting a second maintenance phase pacing escape interval to a rate interval corresponding to the adjusted conduction system pacing rate less the specified time interval.

Example 32. The method of any one of examples 30 or 31 further including sensing ventricular event signals attendant to intrinsic ventricular depolarizations from the at least one cardiac electrical signal and decreasing the conduction system pacing rate by decreasing the first maintenance phase pacing interval in response to sensing at least one ventricular event signal during the maintenance phase.

Example 33. A non-transitory, computer-readable medium storing a set of instructions which, when executed by a control circuit of a medical device, cause the medical device to sense at least one cardiac electrical signal, receive one of a first conduction system pacing therapy command or a second conduction system pacing therapy command, determine an AV pacing interval according to a first method in response to receiving the first conduction system pacing therapy command and determine the AV pacing interval according to a second method different than the first method in response to receiving the second conduction system pacing therapy command. The instructions may further cause the medical device to schedule conduction system pacing pulses for delivery at the determined AV pacing interval and according to the received one of the first conduction system pacing therapy command or the second conduction system pacing therapy command.

Example 34. A medical device system including a user interface configured to receive a user input conduction system pacing therapy command and a display unit configured to display the user interface comprising an auto-populated pacing mode field based on the user input conduction system pacing therapy command. The medical device system may further include a communication circuit for transmitting the user input conduction system pacing therapy command to an implantable medical device configured to deliver a conduction system pacing therapy according to the conduction system pacing therapy command.

Example 35. The medical device system of example 34 wherein the user interface is configured to receive the user input as one of a first conduction system pacing therapy or a second conduction system pacing therapy, and the display unit is further configured to, when the user input is received as the first conduction system pacing therapy, display a maximum atrioventricular conduction time. The display unit may be further configured to, when the user input is received as the second conduction system pacing therapy, display a percentage of an atrioventricular conduction time used by the implantable medical device for determining an atrioventricular pacing interval.

It should be understood that, depending on the example, certain acts or events of any of the methods described herein can be performed in a different sequence, in parallel, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially. In addition, while certain aspects of this disclosure are described as being performed by a single processor, circuit or unit for purposes of clarity, it should be understood that the techniques of this disclosure may be performed by a combination of processors, units or circuits associated with, for example, a medical device system.

In one or more examples, the functions described may be implemented in hardware, software, firmware, or any combination thereof. If implemented in software, the functions may be stored as one or more instructions or code on a computer-readable medium and executed by one or more hardware-based processing units. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. Also, the techniques could be fully implemented in one or more circuits or logic elements.

Thus, a medical device system has been presented in the foregoing description with reference to specific examples. It is to be understood that various aspects disclosed herein may be combined in different combinations than the specific combinations presented in the accompanying drawings. It is appreciated that various modifications to the referenced examples may be made without departing from the scope of the disclosure and the following claims.

1. A medical device, comprising:
a sensing circuit configured to sense at least one cardiac electrical signal;
a therapy delivery circuit configured to generate atrial pacing pulses and conduction system pacing pulses; and
a communication circuit configured to receive a conduction system pacing therapy command; and
a control circuit configured to:
receive the conduction system pacing therapy command from the communication circuit, wherein the conduction system pacing therapy command corresponds to one of at least a first conduction system pacing therapy command or a second conduction system pacing therapy command;
determine an atrioventricular (AV) pacing interval according to a first method when the conduction system pacing therapy command is the first conduction system pacing therapy command; and
determine the AV pacing interval according to a second method different than the first method when the conduction system pacing therapy command is the second conduction system pacing therapy command; and
schedule conduction system pacing pulses for delivery by the therapy delivery circuit at the determined AV pacing interval and according to the received one of the first conduction system pacing therapy command or the second conduction system pacing therapy command.

2. The medical device of claim 1 wherein the control circuit is further configured to:
determine an AV conduction time from the at least one cardiac electrical signal; and
determine the AV pacing interval according to the second method by determining the AV pacing interval as a first percentage of the determined AV conduction time.

3. The medical device of claim 2 wherein the control circuit is further configured to:
compare the AV conduction time to a maximum AV conduction time limit;
determine the AV pacing interval according to the first method by determining the AV pacing interval as a second percentage of the maximum AV conduction time limit; and
when the received conduction system pacing therapy command is the first conduction system pacing therapy command and the AV conduction time is greater than the maximum AV conduction time limit, schedule the conduction system pacing pulses for delivery by the therapy delivery circuit at the AV pacing interval determined as the second percentage of the maximum AV conduction time limit.

4. The medical device of claim 3 wherein the control circuit is further configured to determine the AV pacing interval according to the first method by determining the AV pacing interval as the second percentage of the maximum AV conduction time limit where the second percentage equals the first percentage.

5. The medical device of claim 2 wherein:
the communication circuit is further configured to receive a user programmed AV pacing interval; and
the control circuit is further configured to determine the AV pacing interval according to the first method by determining the AV pacing interval from the received user programmed AV pacing interval.

6. The medical device of claim 1 wherein the control circuit is further configured to:
receive the conduction system pacing therapy command as the first conduction system pacing therapy command;
determine an AV conduction time from the at least one cardiac electrical signal;
determine that the AV conduction time is less than a maximum AV conduction time limit; and
withhold scheduling the conduction system pacing pulses by switching to a single chamber atrial pacing mode when the AV conduction time is less than the maximum AV conduction time limit.

7. The medical device of claim 1 wherein the control circuit is further configured to, in response to the received conduction system pacing therapy command being the first conduction system pacing therapy command:
enable AV conduction checks for detecting AV block;
control the therapy delivery circuit to deliver atrial pacing pulses in a single chamber atrial pacing mode;
switch from the single chamber atrial pacing mode to an atrial synchronous conduction system pacing mode in response to AV conduction block being detected from an AV conduction check; and
schedule the conduction system pacing pulses for delivery by the therapy delivery circuit at the AV pacing interval after switching to the atrial synchronous conduction system pacing mode.

8. The medical device of claim 1 wherein the control circuit is further configured to, in response to the received conduction system pacing therapy command being the second conduction system pacing therapy command:
enable AV conduction checks for measuring AV conduction times;
disable a single chamber atrial pacing mode; and
control the therapy delivery circuit to deliver the conduction system pacing pulses at the AV pacing interval during an atrial synchronous conduction system pacing mode.

9. The medical device of claim 1 wherein the control circuit is further configured to:
receive the conduction system pacing therapy command is as the second conduction system pacing therapy command;
determine an AV conduction time from the at least one cardiac electrical signal;
determine the AV pacing interval as a first percentage of the determined AV conduction time:
determine that the percentage of the AV conduction time is greater than a maximum AV pacing interval limit; and
adjust the AV pacing interval to the maximum AV pacing interval limit in response to the percentage of the AV conduction time is being greater than the maximum AV pacing interval limit.

10. The medical device of claim 1 wherein the control circuit is further configured to:
receive a conduction system pacing therapy command corresponding to a third conduction system pacing therapy command; and
in response to receiving the third conduction system pacing therapy command:
disable AV conduction checks;
disable a single chamber atrial pacing mode; and
schedule conduction system pacing pulses for delivery by the therapy delivery circuit at a programmed AV pacing interval.

11. The medical device of claim 1 wherein the control circuit is further configured to:
determine an atrial rate from at least one of atrial pacing pulses delivered by the therapy delivery circuit or the at least one cardiac electrical signal; and
adjust the AV pacing interval based on the atrial rate.

12. The medical device of claim 1 wherein the control circuit is further configured to:
detect atrial tachyarrhythmia from the at least one cardiac electrical signal;
in response to detecting the atrial tachyarrhythmia, control the therapy delivery circuit to deliver the conduction system pacing pulses at a conduction system pacing rate according to an atrial asynchronous conduction system pacing mode.

13. The medical device of claim 12 wherein the control circuit is further configured to:
determine that at least one of the conduction system pacing pulses delivered at the conduction system pacing rate does not capture cardiac tissue; and
adjust the conduction system pacing rate in response to determining that the at least one conduction system pacing pulse does not capture cardiac tissue.

14. The medical device of claim 12 wherein the control circuit is further configured to:
adjust the conduction system pacing rate during an initialization phase where an initialization phase ending pacing escape interval is reached at which the conduction system pacing pulses are capturing the cardiac tissue;
set a first maintenance phase pacing escape interval to be a specified time interval shorter than the initialization phase ending pacing escape interval; and
for a maintenance time interval, schedule delivery of the conduction system pacing pulses according to the first maintenance phase pacing escape interval.

15. The medical device of claim 14 wherein the control circuit is further configured to:
upon expiration of the maintenance phase, increase the maintenance phase pacing escape interval by the specified time interval to an adjustment phase pacing escape interval;
schedule conduction system pacing pulses at the adjustment phase pacing escape interval;
determine a count of the conduction system pacing pulses that are delivered by the therapy delivery circuit at the adjustment phase pacing escape interval that capture cardiac tissue;
adjust the conduction system pacing rate based on the count; and
set a second maintenance phase pacing escape interval to a rate interval corresponding to the adjusted conduction system pacing rate less the specified time interval.

16. The medical device of claim 14 wherein:
the sensing circuit is further configured to sense ventricular event signals attendant to intrinsic ventricular depolarizations from the at least one cardiac electrical signal; and
the control circuit is further configured to decrease the conduction system pacing rate by decreasing the first maintenance phase pacing interval in response to the sensing circuit sensing at least one ventricular event signal during the maintenance phase.

17. A method comprising:
sensing at least one cardiac electrical signal;
receiving one of a first conduction system pacing therapy command or a second conduction system pacing therapy command;
determining an atrioventricular (AV) pacing interval according to a first method in response to receiving the first conduction system pacing therapy command;
determining the AV pacing interval according to a second method different than the first method in response to receiving the second conduction system pacing therapy command;
scheduling conduction system pacing pulses for delivery at the determined AV pacing interval and according to the received one of the first conduction system pacing therapy command or the second conduction system pacing therapy command; and
delivering a conduction system pacing pulse scheduled at the determined AV pacing interval.

18. The method of claim 17 further comprising:
determining an AV conduction time from the at least one cardiac electrical signal; and
determining the AV pacing interval according to the second method by determining the AV pacing interval as a first percentage of the determined AV conduction time.

19. The method of claim 18 further comprising:
determining that the received conduction system pacing therapy command is the first conduction system pacing therapy command;
comparing the AV conduction time to a maximum AV conduction time limit;
determining that the AV conduction time is greater than the maximum AV conduction time limit;
determining the AV pacing interval according to the first method by determining the AV pacing interval as a second percentage of the maximum AV conduction time limit; and
scheduling the conduction system pacing pulses at the AV pacing interval determined as the second percentage of the maximum AV conduction time limit.

20. A non-transitory, computer-readable medium storing a set of instructions which, when executed by a control circuit of a medical device, cause the medical device to:
sense at least one cardiac electrical signal;
receive one of a first conduction system pacing therapy command or a second conduction system pacing therapy command;
determine an atrioventricular (AV) pacing interval according to a first method in response to receiving the first conduction system pacing therapy command;
determine the AV pacing interval according to a second method different than the first method in response to receiving the second conduction system pacing therapy command;
schedule conduction system pacing pulses for delivery at the determined AV pacing interval and according to the received one of the first conduction system pacing therapy command or the second conduction system pacing therapy command; and
deliver a conduction system pacing pulse that is scheduled at the determined AV pacing interval.
